SEQUENCE LISTING (9 sequences)

SEQ ID NO: 1 - DNA, 20 bp, Severe acute respiratory syndrome-related coronavirus
  source 1..20: mol_type = other DNA
acaccaaaagatcacattgg

SEQ ID NO: 2 - DNA, 20 bp, Severe acute respiratory syndrome-related coronavirus
  source 1..20: mol_type = other DNA
cccgcaatcctgctaacaat

SEQ ID NO: 3 - DNA, 20 bp, synthetic construct
  source 1..20: mol_type = other DNA
acaccaaaagatcacatacc

SEQ ID NO: 4 - DNA, 20 bp, Synthetic construct
  source 1..20: mol_type = other DNA
acaccaaactctcacattgg

SEQ ID NO: 5 - DNA, 20 bp, Synthetic construct
  source 1..20: mol_type = other DNA
acaccaaaagaggacattgg

SEQ ID NO: 6 - DNA, 20 bp, synthetic construct
  source 1..20: mol_type = other DNA
acttcaaaagatcacattgg

SEQ ID NO: 7 - DNA, 20 bp, synthetic construct
  source 1..20: mol_type = other DNA
acttcaaaagaggacattgg

SEQ ID NO: 8 - DNA, 20 bp, synthetic construct
  source 1..20: mol_type = other DNA
acttcaaaagaggacatacc

SEQ ID NO: 9 - DNA, 20 bp, synthetic construct
  source 1..20: mol_type = other DNA
acaccaaaagaggagtcacc

(19) United States
(12) Patent Application Publication
(10) Pub. No.: US 2025/0003017 A1
(43) Pub. Date: Jan. 2, 2025

(54) Antisense Oligonucleotides Guided Hyperspectral Nanoprobes for Rapid Point-of-Care Molecular Diagnosis of Emerging Infectious Diseases

(71) Applicants: The Penn State Research Foundation, University Park, PA (US); University of Maryland, Baltimore, Baltimore, MD (US); University of Marlyand Baltimore County, Baltimore, MD (US)

(72) Inventors: Dipanjan Pan, Ellicott City, MD (US); Parikshit Moitra, Baltimore, MD (US); Maha Mohammad Shehadeh Alafeef, Baltimore, MD (US)

(21) Appl. No.: 18/758,644

(22) Filed: Jun. 28, 2024

Related U.S. Application Data
(60) Provisional application No. 63/524,020, filed on Jun. 29, 2023.

(51) Int. Cl. C12Q 1/70; C12Q 1/6834; C12Q 1/6876

(52) U.S. Cl. CPC C12Q 1/701; C12Q 1/6834; C12Q 1/6876; C12Q 2600/156

(57) ABSTRACT

Embodiments relate to compositions, methods, and systems for screening and detecting respiratory diseases. In particular, embodiments relate to a hyperspectral image-based assay configured to simultaneously detect the presence of one or more target gene sequences of respiratory diseases of interest. Embodiments may utilize antisense oligonucleotides designed specifically to bind in complementary fashion to a target gene sequence of a respiratory disease of interest.

### CROSS-REFERENCE TO RELATED APPLICATIONS

This patent application is related to and claims the benefit of priority of U.S. Provisional Application No. 63/524,020, filed on Jun. 29, 2023, the entire contents of which is incorporated by reference.

### INCORPORATION BY REFERENCE STATEMENT REGARDING SEQUENCE LISTINGS

A Sequence Listing using exXtensible Markup Language (XML) compliant with World Intellectual Property Organization (WIPO) Standard ST.26 is provided herewith and the entirety of this sequence listing is incorporated by reference herein. The Sequence Listing that is incorporated by reference herein is e-filed at the USPTO using Patent Center as an XML file, named 0073605-000864.xml (9 KB in size, created on Jun. 27, 2024).

### FIELD

Embodiments relate to apparatuses, methods, and systems for screening and detecting respiratory diseases. In particular, embodiments may relate to hyperspectral imaging systems configured to simultaneously detect and distinguish between multiple respiratory diseases and their variants.

### BACKGROUND

The coronavirus disease 2019 (COVID-19) was caused by a novel severe acute respiratory syndrome-coronavirus-2 (SARS-COV-2). A crucial shortcoming of healthcare systems across the globe has been the ability to rapidly and accurately detect co-infection with other respiratory diseases, with contributing factors such as shortages of test kits and specimen materials, and no established funding mechanism to support testing facilities. Furthermore, current gold standard tests, such as reverse transcription-polymerase chain reaction (RT-PCR) tests, for diagnosing infection due to SARS-COV-2 are time-consuming, complex, and labor-intensive, requiring each sample to be sent to a laboratory for confirmation. For example, most current screening techniques require sampling of body fluids such as nasal fluid, saliva, or blood, followed by nucleic acid-based testing to identify active infections or blood-based serological identification of past infections. Although these techniques may be highly sensitive, nucleic acid-based diagnostics may require samples gathered several days post-exposure for unambiguous positive detection. Moreover, a “positive” RT-PCR test outcome indicates only the identification of viral RNA and does not generally mean that a viable virus is present. This could potentially lead to missed asymptomatic cases. Therefore, the ability to perform practical real-time COVID-19 screening is extremely crucial in assessing the risk and monitor pathogen presence in the community.

Additionally, the COVID-19 virus is constantly changing, leading to the emergence of variants with new characteristics. Although vaccines continue to reduce a person's risk of contracting the virus that cause COVID-19, emerging variants can impact their effectiveness. Community transmission of COVID-19 can stimulate new mutations in the virus, potentially resulting in more virulent strains with higher mortality rates or fast transmission speed. Therefore, it is undeniable that systematic tracking of strain information is essential to effectively mitigate COVID-19.

### SUMMARY

We determined that there is an ongoing need for a rapid, cost-effective, and selective diagnostic test for COVID-19 and to differentiate COVID-19 from the other common respiratory pathogens. We have therefore designed a universal platform for pathogen detection that utilizes a hyperspectral-based nano-imaging technique for rapid multiplexed detection that may distinguish between respiratory diseases and their variants.

In the development of a POC diagnostic test for rapid detection of respiratory diseases, a crucial step involves the identification of reliable sensing probes that offer selective and sensitive detection of specific genetic targets in the respiratory diseases. Single-stranded oligonucleotides (ssDNAs), which are fragments of nucleic acids, emerge as valuable tools in this process. Accordingly, we have designed a series of oligonucleotide probes targeting different genetic segments of respiratory diseases and their variants, for use in the hyperspectral-based nano-imaging system. The probes may be conjugated onto the surface of nanoparticles, which may selectively aggregate as the probes bind to their target genes sequences (e.g., DNA from one of any number of respiratory diseases of interest). Such aggregation may be detected to determine the presence of a respiratory disease. The designed oligonucleotide probes provide a solution for streamlining quantitative detection and variant classification of respiratory pathogens using a rapid (e.g., less than 10 minutes sample to test results) and inexpensive nano-sensor panel.

Embodiments may be configured for simultaneous detection and identification of nucleic acids from multiple respiratory viruses or from multiple variants of a respiratory virus collected from individuals with clinical signs and symptoms of a respiratory tract infection. Overall, embodiments provide a highly sensitive functional assay for the diagnosis of multiple respiratory pathogens from a single platform and with RT-PCR level accuracy and high throughput screening but within a fraction of the time needed by existing techniques.

In an exemplary embodiment, a hyperspectral-based imaging apparatus for detecting one or more variants of a respiratory disease in a sample comprises a first sensing probe functionalized with a moiety at its three prime end, wherein the first sensing probe has a sequence that is complementary of a first target gene sequence of the respiratory disease; a second sensing probe functionalized with a moiety at its five prime end, wherein the second sensing probe has a sequence that is complementary of a second target gene sequence of the respiratory disease; a third sensing probe functionalized with a moiety at its five prime end, wherein the third sensing probe has a sequence that is complementary of a third target gene sequence of the respiratory disease, wherein the third target gene sequence is a mutation of the second target gene sequence; and a plurality of nanoparticles bound to the moieties of the first, second, and third sensing probes, wherein upon the first and second sensing probes binding to the first and second target gene sequences of the respiratory disease, the nanoparticles are brought within proximity of one another and agglomerate, and wherein upon the first and third sensing probes binding to the first and third target gene sequences of the respiratory disease, the nanoparticles are brought within proximity of one another and agglomerate.

In some embodiments, the mutation of the second target gene sequence is selected from the group consisting of a single point mutation, a dual point mutation, and a triple point mutation.

In some embodiments, the respiratory disease is SARS-COV-2.

In some embodiments, the first target gene sequence is SEQ ID No. 2 (CCCGCAAUCCUGCUAACAAU).

In some embodiments, the second target gene sequence is SEQ ID NO. 1 (ACACCAAAAGAUCACAUUGG).

In some embodiments, the third target gene sequence is selected from the group consisting of SEQ ID NO. 3 (ACACCAAAAGAUCACAUACC), SEQ ID NO. 4
(ACACCAAACUCUCACAUUGG), SEQ ID NO. 5 (ACACCAAAAGAGGACAUUGG), SEQ ID NO. 6 (ACUUCAAAAGAUCACAUUGG), SEQ ID NO. 7
(ACUUCAAAAGAGGACAUUGG), SEQ ID NO. 8 (ACUUCAAAAGAGGACAUACC), and SEQ ID NO. 9 (ACACCAAAAGAGGAGUCAC).

In some embodiments, the apparatus further comprises a fourth sensing probe functionalized with a moiety at its five prime end, wherein the fourth sensing probe has a sequence that is complementary of a fourth target gene sequence of the respiratory disease, wherein the fourth target gene sequence is a mutation of the second target gene sequence; and nanoparticles bound to the moiety of the fourth sensing probes, wherein upon the first and fourth sensing probes binding to the first and fourth target gene sequences of the respiratory disease, the nanoparticles are brought within proximity of one another and agglomerate.

In some embodiments, the apparatus further comprises a fifth sensing probe functionalized with a moiety at its five prime end, wherein the fifth sensing probe has a sequence that is complementary of a fifth target gene sequence of the respiratory disease, wherein the fifth target gene sequence is a mutation of the second target gene sequence; and nanoparticles bound to the moiety of the fifth sensing probes, wherein upon the first and fifth sensing probes binding to the first and fourth target gene sequences of the respiratory disease, the nanoparticles are brought within proximity of one another and agglomerate.

In some embodiments, the apparatus further comprises a sixth sensing probe functionalized with a moiety at its five prime end, wherein the sixth sensing probe has a sequence that is complementary of a sixth target gene sequence of the respiratory disease, wherein the sixth target gene sequence is a mutation of the second target gene sequence; a seventh sensing probe functionalized with a moiety at its five prime end, wherein the seventh sensing probe has a sequence that is complementary of a seventh target gene sequence of the respiratory disease, wherein the seventh target gene sequence is a mutation of the second target gene sequence; an eighth sensing probe functionalized with a moiety at its five prime end, wherein the eighth sensing probe has a sequence that is complementary of a eighth target gene sequence of the respiratory disease, wherein the eighth target gene sequence is a mutation of the second target gene sequence; a ninth sensing probe functionalized with a moiety at its five prime end, wherein the ninth sensing probe has a sequence that is complementary of a ninth target gene sequence of the respiratory disease, wherein the ninth target gene sequence is a mutation of the second target gene sequence; and a plurality of nanoparticles bound to the moieties of the sixth, seventh, eighth, and ninth sensing probes, wherein upon the first and sixth sensing probes binding to the first and second target gene sequences of the respiratory disease, the nanoparticles are brought within proximity of one another and agglomerate, wherein upon the first and seventh sensing probes binding to the first and third target gene sequences of the respiratory disease, the nanoparticles are brought within proximity of one another and agglomerate; wherein upon the first and eighth sensing probes binding to the first and second target gene sequences of the respiratory disease, the nanoparticles are brought within proximity of one another and agglomerate, and wherein upon the first and ninth sensing probes binding to the first and second target gene sequences of the respiratory disease, the nanoparticles are brought within proximity of one another and agglomerate.

In some embodiments, the moiety is selected from a thiol moiety and an amino moiety.

In some embodiments, the nanoparticles are selected from gold nanoparticles and hafnium nanoparticles.

In some embodiments, the nanoparticles are hafnium nanoparticles.

In some embodiments, the apparatus further comprises a test panel comprising a sample inlet region and a sensing region, wherein the first, second, and third sensing probes are deposited at or near the sensing region, and wherein the sample inlet region is configured to receive the sample, which is configured flow through the test panel towards the sensing region; and a hyperspectral imaging sensor configured to receive the test panel.

In some embodiments, the hyperspectral imaging sensor is further configured to capture hyperspectral images of the test panel.

In an exemplary embodiment, a hyperspectral imaging-based method for detecting one or more variants of a respiratory disease in a sample comprises providing a test panel. The test panel comprises a sample inlet region, a sensing region, and a plurality of sensing probes deposited at or near the sensing region, the plurality of sensing probes. The plurality of sensing probes comprise a first sensing probe functionalized with a moiety at its three prime end, wherein the first sensing probe has a sequence that is complementary of a first target gene sequence of the respiratory disease; a second sensing probe functionalized with a moiety at its five prime end, wherein the second sensing probe has a sequence that is complementary of a second target gene sequence of the respiratory disease; a third sensing probe functionalized with a moiety at its five prime end, wherein the third sensing probe has a sequence that is complementary of a third target gene sequence of the respiratory disease, wherein the third target gene sequence is a mutation of the second target gene sequence; and a plurality of nanoparticles bound to the moieties of the first, second, and third sensing probes, wherein upon the first and second sensing probes binding to the first and second target gene sequences of the respiratory disease, the nanoparticles are brought within proximity of one another and agglomerate, and wherein upon the first and third sensing probes binding to the first and third target gene sequences of the respiratory disease, the nanoparticles are brought within proximity of one another and agglomerate. The method further comprises integrating the test panel with a hyperspectral imaging sensor configured to capture hyperspectral images of the test panel.

In some embodiments, the method further comprises integrating the hyperspectral imaging sensor with an external device configured to display and/or store and/or analyze the captured hyperspectral images.

In some embodiments, the respiratory disease is SARS-COV-2.

In some embodiments, the first target gene sequence is SEQ ID NO. 2.

In some embodiments, the second target gene sequence is SEQ ID NO. 1.

In some embodiments, the third target gene sequence is selected from the group consisting of SEQ ID NO. 3, SEQ ID NO. 4, SEQ ID NO. 5, SEQ ID NO. 6, SEQ ID NO. 7, SEQ ID NO. 8, and SEQ ID NO. 9.

### BRIEF DESCRIPTION OF THE DRAWINGS

The above and other objects, aspects, features, advantages, and possible applications of embodiments of the present innovation will be more apparent from the following more particular description thereof, presented in conjunction with the following drawings. Like reference numbers used in the drawings may identify like components.

FIG. 1 is a schematic illustration showing an exemplary hyperspectral image-based apparatus, system, and method.

FIG. 2 is a schematic illustration showing exemplary sensing probes differentially functionalized with nanoparticles and targeting respective gene sequences.

FIG. 3 is a schematic diagram showing the development of an exemplary sensor.

FIG. 4 is a schematic illustration showing mutation sites of gene sequences and their alignment with exemplary sensing probes.

FIG. 5 is a schematic illustration showing an exemplary hyperspectral image-based sensor.

FIG. 6 is a schematic showing conjugation chemistry of an exemplary sensing probe and an exemplary sensing mechanism of the conjugated probe.

FIG. 7 is a block diagram illustrating an exemplary system for implementing the sensor.

FIG. 8 is a diagram of highest occupied molecular orbitals (HOMO) and least unoccupied molecular orbitals (LUMO) for (a, c) ASO1 and (b, d) ASO2 conjugated (a, b) Hf and (c, d) Au nanoparticles while bonded in their docked geometry with the target SARS-COV-2 RNA sequence.

FIG. 9 is a graph showing change in absorbance at 600 nm of ASO conjugated HfNPs upon addition of SARS-COV-2 RNA.

FIG. 10 is enhanced darkfield hyperspectral imaging (EDF-HSI) for HfNPs-Pₘᵢₓ, and AuNPs-Pₘᵢₓ respectively after the addition of SARS-COV-2 RNA.

FIG. 11 is a graph showing a shift in the hyperspectral signal as obtained from the AuNPs and HfNPs as a sensing probe (n=3, P<0.05).

FIG. 12 is a graph showing the effect of different mediums on particle size.

FIG. 13 is a graph showing the effect of temperature on particle size.

FIG. 14 is an NMR spectra of HfNP, HfNP conjugated ASO (HfNPs-P₁), and HfNP conjugated ASO (HfNPs-P₂), respectively.

FIG. 15 is a schematic illustration showing an exemplary method and workflow to find the predominant spectral signature.

FIG. 16 shows hyperspectral mapping of a representative COVID-19 positive clinical sample.

FIG. 17 shows hyperspectral mapping image of the spectral component of the image superimposed on the enhanced dark-field image of COVID-19 positive sample (Ct number=18.9),

FIG. 18 shows hyperspectral mapping image of the spectral component of the image superimposed on the enhanced dark-field image of COVID-19 negative sample. Formation of large clusters can be observed.

FIG. 19 shows predominant spectral signature in both positive and negative samples. The positive sample showed a significant peak shift when compared with the negative sample.

FIG. 20 shows EDF-HSI of ssDNA-conjugated to HfNPs (HfNPs-Pₘᵢₓ) in the presence of SARS-COV-2 RNA with concentration a, 97.2fM, b, 97.21zM, c, 0.09721 yM. The presence of SARS-COV-2 RNA leads to the aggregation of HfNPs to form large entities. e, EDF-HSI of the ssDNA-conjugated to HfNPs (HfNPS-Pₘᵢₓ) in the absence of the target, f, in the presence of SARS-COV-2 RNA at very low concentration ˜0.1yM. 4X: amplification of four times of 100× oil objective. The sensor enables the detection of SARS-COV-2 RNA at a very low concentration of ˜0.1 yM.

FIG. 21 is a graph showing a standard curve of the sensor which shows that the SARS-CoV-2 Log₁₀ (RNA) is linearly proportional with the peak shift, Pearson's correlation=0.94, R2=0.88.

FIG. 22 is a graph showing hyperspectral signal of the HfNPs-Pₘᵢₓ in the absence and presence of SARS-COV-2 RNA (0.09721yM). A significant shift in the signal peak can be observed after the addition of a sample containing SARS-COV-2 genetic material. The experiments were performed with experimental repeats of n=8.

FIG. 23 is a graph showing hydrodynamic diameters of the HfNPs-Pₘᵢₓ in the presence of SARS-COV-2 and MERS-COV RNA (n=4, P<0.001).

FIG. 24 shows EDF-HSI of the HfNPs-Pₘᵢₓ in the presence of SARS-COV-2 genomic RNA.

FIG. 25 shows a TEM image of HfNPs-Pₘᵢₓ.

FIG. 26 shows TEM images of HfNPs-Pₘᵢₓ in the presence of SARS-COV-2 RNA.

FIG. 27 shows EDF-HSI of the HfNPs-Pₘᵢₓ in the presence of MERS-COV, SARS-COV, Influenza A H1N1, and SARS-COV-2 RNA from COVID-19 confirmed clinical sample.

FIG. 28 shows EDF-HSI of the HfNPs-Pₘᵢₓ after the addition of confirmed COVID-19 negative sample, and confirmed COVID-19 positive sample.

FIG. 29 is a graph showing the presence of SARS-COV-2 genetic material leads to the formation of large entities. A significant shift in the peak wavelength was observed after the addition of the confirmed COVID-19 positive sample.

FIG. 30 is a graph showing individual values of the hyperspectral peak shift obtained from the system as a response to the 66 clinical samples with their cycle threshold number (Ct number) obtained from RT-PCR. The peak shift of the positive samples is linearly inversely proportional with the samples Ct number, Pearson's correlation=−0.8928, R2=0.86, X intercept>40 Ct number; indicating its sensitivity.

FIG. 31 is a column plot of the peak shift in nm of the sensor as a response to 66 COVID-19 clinical samples.

FIG. 32 is a confusion matrix comparing the classification results of the sensor as benchmarked to the standard RT-PCR.

FIG. 33 is graph showing a comparison of the limit of detection obtained using the sensor and other available COVID-19 tests. EUA: emergency used authorization from the food and drug administration. The experiments were performed with experimental repeats of n=8.

FIG. 34 is a schematic representation of the sensor to test Influenza A H1N1 sample.

FIG. 35 shows EDF-HSI of the HfNPs-Pₘᵢₓ in the absence of samples, and after the addition of Influenza A H1N1 sample.

FIG. 36 is a schematic representation of the sensor to test samples lacking influenza A H1N1 RNA.

FIG. 37 is a graph showing peak shift in nm after the addition of SARS-COV-2, H11N1 A, H1N1 B, H1N1 MD, and SARS-COV viruses respectively.

FIG. 38 shows spectra collected using the hyperspectral technique for the unbound HfNPs functionalized with ASOs specific to the Influenza A H1N1 genetic materials in the presence of H1N1 A sample, SARS-COV, H1N1 MD, H1N1 B, and SARS-COV-2.

FIG. 39 shows EDF-HSI after the addition of influenza B H1N1 sample, influenza MD H1N1 (Maryland strain) sample, SARS-COV sample, and SARS-COV-2 sample. The experiments were performed with experimental repeats of n=8.

FIG. 40 is a schematic showing clinical samples in VTM were passed through the NAP-10 column and mixed directly with the HfNPs-Pₘᵢₓ.

FIG. 41 is a graph showing a peak shift in the HSI signal as a response to 33 COVID-19 clinical samples as a function of their Ct value as obtained from RT-PCR. Pearson's correlation was found to be −0.8. The positive and the negative COVID-19 samples were correctly distinguished from each other (threshold=11).

FIG. 42 is a graph showing a receiver operating characteristic (ROC) curve.

FIG. 43 is a graph showing a column plot of the peak shift in nm obtained from the sensor as a response to two groups, positive and negative COVID-19 samples.

FIG. 44 shows EDF-HSI of the COVID-19 positive and negative samples. Large entities were formed when a positive sample was tested.

FIG. 45 shows a confusion matrix comparing the classification results of the sensor as benchmarked to the standard RT-PCR of all the tested clinical samples. The experiments were performed with experimental repeats of n=8.

### DETAILED DESCRIPTION

The following description is of exemplary embodiments and methods of use that are presently contemplated for carrying out the present invention. This description is not to be taken in a limiting sense, but is made merely for the purpose of describing the general principles and features of various aspects of the present invention. The scope of the present invention is not limited by this description.

Embodiments generally relate to apparatuses, methods, and systems configured to accurately screen and detect respiratory diseases and variants of respiratory diseases. Exemplary compositions may comprise sensing probes configured to selectively detect a target gene sequence of a respiratory disease.

### Hyperspectral-Based Imaging (HSI) Assay

Embodiments may relate to an HSI apparatus and system configured to receive and analyze a sample to determine if the sample comprises target gene sequences related to respiratory diseases and/or variants of respiratory diseases. The HSI apparatus and system can be used as a point-of-care (POC) test, for example as a rapid lab test, for screening and detection of respiratory diseases. It is contemplated that the term “respiratory diseases” as used herein should be understood to encompass both different respiratory diseases as well as variants of a single respiratory disease.

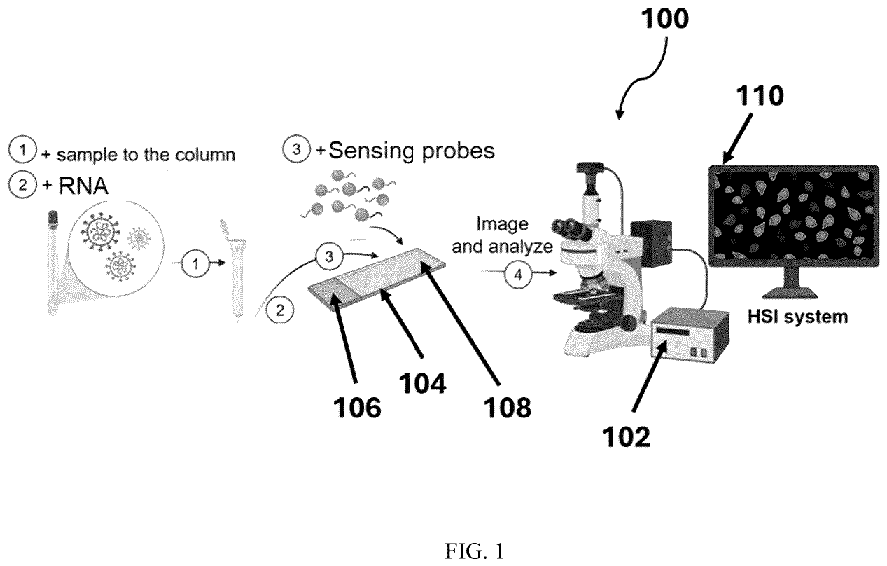

As seen in FIG. 1, a system 100 may comprise a HSI sensor 102 configured to receive a test panel 104 for the detection of respiratory diseases from a collected sample. The test panel 104 may comprise a sample inlet region 106 at or near a first end of the panel 104 and a sensing region 108 at or near an opposite second end of the panel 104. It is contemplated that a sample collected from a subject, or a solution comprising a sample collected from a subject, may be placed on or at the sample inlet region 106 and flow through the panel 104 (e.g., across the length of the panel 104) towards the sensing region 108 thereafter. In some embodiments, the panel 104 may be configured as a disposable (e.g., one-time use) panel.

In exemplary embodiments, the sensing region 108 comprises sensing probes. In particular, anti-sense oligonucleotides (ASOs) may be configured as sensing probes to detect one or more respiratory diseases present in a sample. The ASOs are single-stranded DNA (ssDNA) probes designed specifically to bind in complementary fashion to a target gene sequence of a respiratory disease of interest. For example, ASOs have nucleotide sequences that complement the nucleotide sequence of a target gene (e.g., adenine (A) in an ASO sequence may complement and bind to uracil (U) or thymine (T) in a target gene sequence, cytosine (C) in an ASO sequence may complement and bind to guanine (G) in a target gene sequence, thymine (T) or uracil (U) in an ASO sequence may complement and bind to adenine (A) in a target gene sequence, and guanine (G) in an ASO sequence may complement and bind to cytosine (C) in a target gene sequence). It is contemplated that the terms anti-sense oligonucleotides, single-stranded DNA, and sensing probes may be used interchangeably herein.

The sensing probes may be used to form capped nanoparticles. It is contemplated that a “capped” nanoparticle means a nanoparticle that is covalently bound to another molecule, such as to a sensing probe. Sensing probes may be functionalized with a moiety at either their first end or their second end such that the functionalized ends may bind to the surface of the nanoparticles via the moiety. It is contemplated that the first end can be a five prime end (5′ end) and the second end can be a three prime end (3′ end). It is contemplated that the moiety may be selected from a thiol group (—SH) and an amino group (—NH₂). As the moieties are coupled to both the sensing probes and the nanoparticles, when the sensing probes bind to a target gene sequence, the nanoparticles are also necessarily present at the target gene sequence.

The nanoparticles may be gold nanoparticles or hafnium nanoparticles, or may be nanoparticulates that include gold or hafnium. In preferred embodiments, the nanoparticles may be hafnium nanoparticles or are nanoparticulates that include hafnium. Other embodiments can utilize other types of suitable particulates. It is contemplated that the sensing probes may be functionalized with a thiol moiety when the nanoparticles are gold nanoparticles or nanoparticulates that include gold, and the sensing probes may be functionalized with an amino moiety when the nanoparticles are hafnium nanoparticles or nanoparticulates that include hafnium.

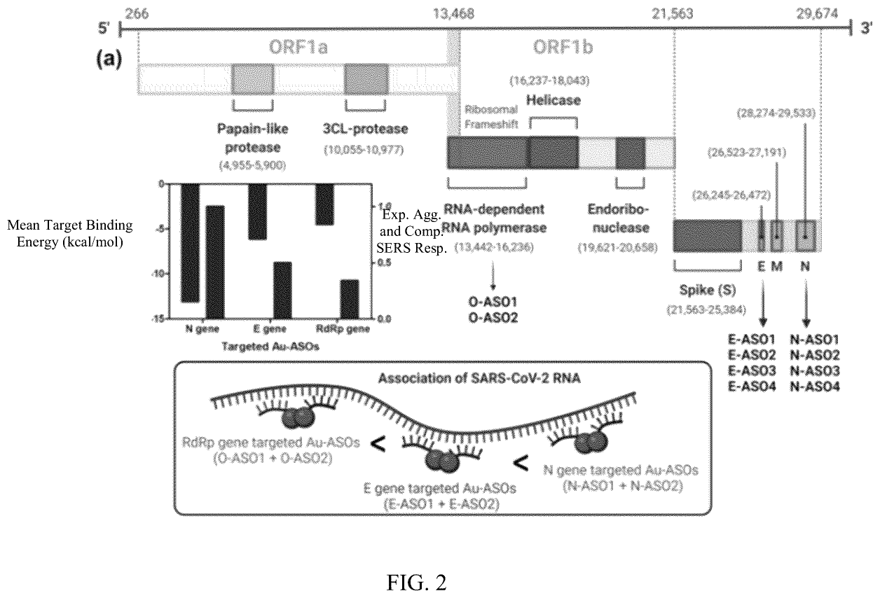

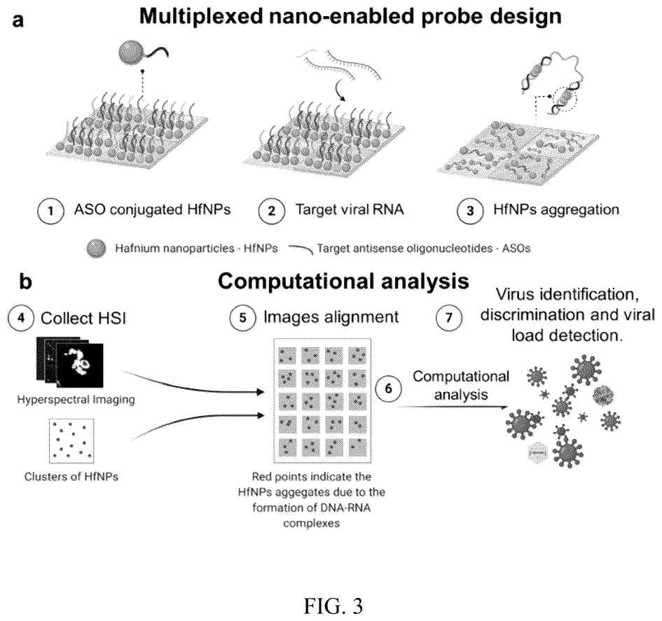

In exemplary embodiments, sensing probes may be chosen in pairs and may be configured to bind to two closely spaced regions of a target gene sequence. It is contemplated that each sensing probe of the pair may be differentially functionalized (e.g., the first sensing probe functionalized at its first end and the second sensing probe functionalized at its second end) such that the functionalized ends of each sensing probe are in close proximity to each other when the sensing probes are bound to their respective regions of the target gene sequence (see FIGS. 2-3). For example, a first sensing probe functionalized at its first end may be complementary of a first region of a target gene sequence, and a second sensing probe functionalized at its second end may be complementary of a second region of a target gene sequence that is in close proximity to the first region. As the functionalized ends of the sensing probes may bind to nanoparticles, the nanoparticles necessarily come close to each other when the sensing probes binds to their respective regions of the target gene sequence. This results in an agglomeration of nanoparticles, which may be observed and detected as is further described below, and the presence of the respiratory disease corresponding to the target gene sequence may therefore be detected.

It is contemplated that when sensing probes are not in the presence of their target gene sequence, the sensing probes may be individually dispersed and an agglomeration of nanoparticles will not be detected. However, when sensing probes are in the presence of their target gene sequence, the nanoparticles may agglomerate to form large, detectable clusters, and the presence of the respiratory disease corresponding to the target gene sequence may therefore be detected. Accordingly, the sensing probes exhibit selective aggregation in the presence of their target gene sequence.

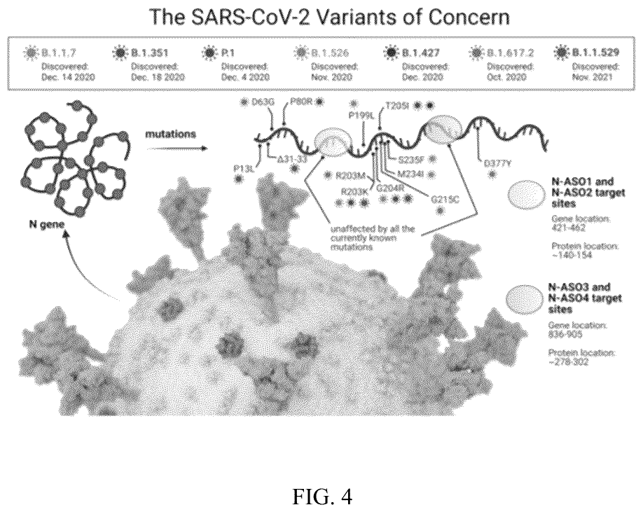

It is contemplated that the sensing probes may be designed to target gene sequences that are less prone to mutation and/or antibiotic resistance (see FIG. 4). For example, ASOs may be designed to target regions that may be conserved among different strains of a respiratory disease and less prone to antibiotic resistance. By targeting conserved regions, ASOs may be used universally for diagnostic purposes, ensuring consistent and reliable results regardless of genetic mutations among strains.

In exemplary embodiments, the sensing region 108 may comprise a plurality of sensing probe pairs and/or an array of sensing probe pairs. It is contemplated that the sensing region 108 can include any number of sensing probe pairs, such as between 2-50 sensing probe pairs, 4-40 sensing probe pairs, 5-30 sensing probe pairs, 6-20 sensing probe pairs, 8-15 sensing probe pairs, though the number of sensing probe pairs is not particularly limited.

The sensing region 108 may comprise at least a first sensing probe pair configured to complement and bind to a first target gene sequence, a second sensing probe pair configured to complement and bind to a second target gene sequence, a third sensing probe pair configured to complement and bind to a third target gene sequence, a fourth sensing probe pair configured to complement and bind to a fourth target gene sequence, etc. It is contemplated that the different target gene sequences may correspond to gene sequences of different respiratory diseases. For example, the first target gene sequence may correspond to a first respiratory disease, the second target gene sequence may correspond to a second respiratory disease, the third target gene sequence may correspond to a third respiratory disease, the fourth target gene sequence may correspond to a fourth respiratory disease, etc. Accordingly, a single system may be used for simultaneously screening and detecting various different respiratory diseases.

It is further contemplated that the different target gene sequences may correspond to gene sequences of different variants of at least one respiratory disease. For example, a first target gene sequence may correspond to a first variant of a respiratory disease, a second target gene sequence may correspond to a second variant of a respiratory disease, a third target gene sequence may correspond to a third variant of a respiratory disease, a fourth target gene sequence may correspond to a fourth variant of a respiratory disease, etc. Accordingly, a single system may be used for simultaneously screening and detecting different variants of at least one respiratory disease.

In some embodiments, the sensing region 108 may comprise an array of sensing probe pairs wherein the array includes a plurality of sections. Individual sections may correspond to different respiratory diseases. For example, a first section may include first sensing probe pairs configured to detect a first respiratory disease, a second section may include second sensing probe pairs configured to detect a second respiratory disease, a third section may include third sensing probe pairs configured to detect a third respiratory disease, a fourth section may include fourth sensing probe pairs configured to detect a fourth respiratory disease, etc. Individual sections may also correspond to different variants of a respiratory disease. For example, a first section may include first sensing probe pairs configured to detect a first variant of a respiratory disease, a second section may include second sensing probe pairs configured to detect a second variant of a respiratory disease, a third section may include third sensing probe pairs configured to detect a third variant of a respiratory disease, a fourth section may include fourth sensing probe pairs configured to detect a fourth variant of a respiratory disease, etc.

It is contemplated that the sensing region 108 can be configured to detect one or more respiratory diseases, including but not limited to Influenza A, Rhinovirus/Enterovirus, Adenovirus, Parainfluenza virus 1, Coronavirus HKU1, Influenza A H3, Parainfluenza virus 2, Coronavirus NL63, Influenza B, Parainfluenza virus 3, Coronavirus 229E, Respiratory Syncytial Virus A, Parainfluenza virus 4, Coronavirus OC43, Respiratory Syncytial Virus B, Human Metapneumovirus, Human Bocavirus, MERS, SARS-COV-2, Chlamydophila pneumoniae, Mycoplasma pneumonia, Legionella pneumonia, and Influenza A H1N1. Put differently, the sensing region 108 can include sensing probes configured to complement and bind to a target gene sequence corresponding to each of these diseases.

It is contemplated that the sensing region 108 can be configured to detect one or more variants of SARS-COV-2, such as Alpha, Beta, Gamma, Epsilon, Eta, Iota, Kappa, Mu, Zeta, Delta, and Omicron. Put differently, the sensing region 108 can include sensing probes configured to complement and bind to a target gene sequence and sensing probes configured to complement and bind to mutations (e.g., single point, dual point, and triple point mutations) of that target gene sequence. This ensures that a respiratory disease of interest is detected even if its target gene sequences undergoes known mutations.

It is contemplated that the sensing probes may be deposited on the test panel 104 at the sensing region 108 via an inkjet bioprinter. In particular, a solution comprising the sensing probes may be prepared, and the solution may then be deposited.

As seen in FIG. 1, exemplary methods and systems for screening and detecting respiratory diseases may comprise collecting a sample (e.g., an RNA/DNA sample) from a subject. It is contemplated that the sample may be collected using any suitable means, including but not limited to, an oral swab, a nasal swab, a cervical swab, a blood collecting swab, urine collection, or any other suitable means for collecting nucleic acid from the subject. It is further contemplated that the sample may be collected using any suitable instrument, including but not limited to, a cotton swab or any other suitable instrument for collecting nucleic acid from the subject.

The collected sample may then be introduced to a solution to form an aqueous sensing solution. After the sensing solution is obtained, the sensing solution may be placed at or near the sample inlet region 106. The aqueous sensing solution may then flow through the test panel 104 in a flow direction and towards the sensing region 108. When the sensing solution comprises a target gene sequence, the sensing probes deposited at the sensing region 108 may bind to their respective target gene sequences, and nanoparticles functionalized on the sensing probes may agglomerate to form large, detectable clusters. On the contrary, when the sensing solution does not comprise a target gene sequence, an agglomeration of nanoparticles will not form.

It is contemplated that the aggregation of sensing probes/nanoparticles can be detected by hyperspectral imaging (HSI). Generally, HSI is configured to detect scattering of light caused by the aggregation of sensing probes/nanoparticles, which may then be used to estimate concentration of target gene sequences. More specifically, HSI is capable of identifying individual nanoparticles in suspensions and in the presence of biological samples by their intrinsic scattering spectra without the use of any labeling agent. HSI is a sensitive technique that analyzes a wide spectrum of light to obtain information that is not available when imaging with primary colors (red, green, blue). A shift in the HSI peak can be observed due to the aggregation of sensing probes/nanoparticles.

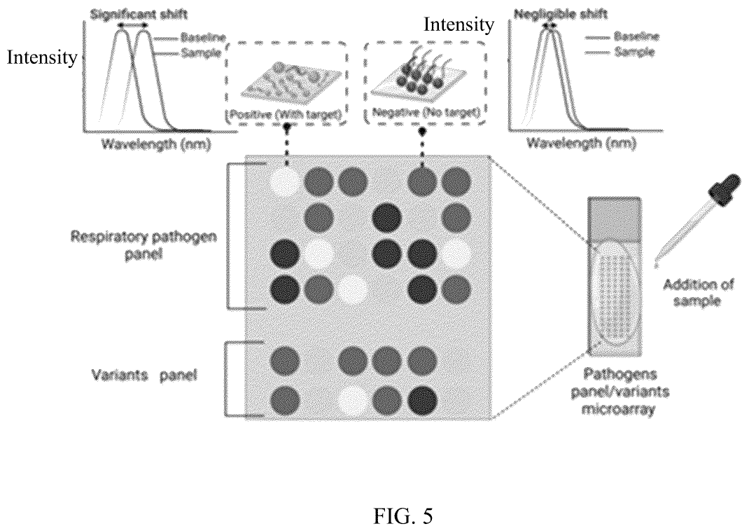

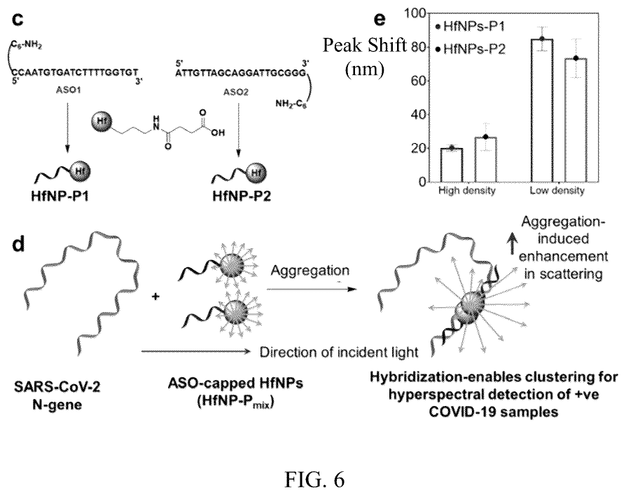

After the sensing solution is applied to the test panel 102, the HSI sensor 102 may receive the test panel 102, particularly the sensing region 108 of the test panel 102. The HSI sensor 102 is configured to capture HSI images of the sensing region 108 to detect aggregations of nanoparticles, which signals the presence of at least one respiratory disease present in the sample. In particular, as seen in FIGS. 5-6, the HSI sensor 102 may detect the aggregation of nanoparticles via a shift in light scattering of the nanoparticles. The captured HSI images and their hyperspectral signals may be analyzed to determine the peak wavelength (in nm), and the shift in the peak of the sample compared to a reference sample may be calculated and used for virus identification.

As can be appreciated by the above, the system 100 may be configured to detect the presence of one or more respiratory diseases in a collected sample. The system 100 may therefore serve as a one step, simultaneous detection method for various respiratory diseases in a POC setting.

As there is an ongoing and immediate need to develop approaches that are low-cost, rapid, and can be used as a screening tool for the diagnosis of respiratory diseases at POC, it is contemplated that embodiments described herein may provide one or more advantages over currently available screening and detecting techniques. For example, embodiments described herein do not need prior RNA extraction, and/or have short turnaround times. In some embodiments, the presently described system provides for rapid turnaround time for detection of one or more respiratory diseases. The detection of a respiratory disease of interest may be performed within about 5, 10, 15, 20, 25, or 30 minutes. In one embodiment, detection may be completed within about 10 minutes.

The system 100 may also have a limit of detection in the yoctomolar range, which is 1,000,000-fold times higher than the currently available tests. It is contemplated that the system 100 may be highly sensitive and have a specificity of 100%, which indicates that the system 100 can be used both with presymptomatic and asymptomatic patients.

It is contemplated that detection of a disease can be made via the HSI sensor 102 or via a first external device 110 (e.g., tablet, smart phone, laptop computer, personal computer) that can receive data from the HSI sensor 102 and analyze the data to determine whether target gene sequences are present. The results of the data analysis from the sample can be output via a display on the HSI sensor 102 and/or on the first external device 110, and/or via a printer for outputting the result of the conducted testing. The HSI sensor 102 may be communicatively connectable to the first external device 110 via a network connection (e.g., internet connection, wide area network connection, etc.).

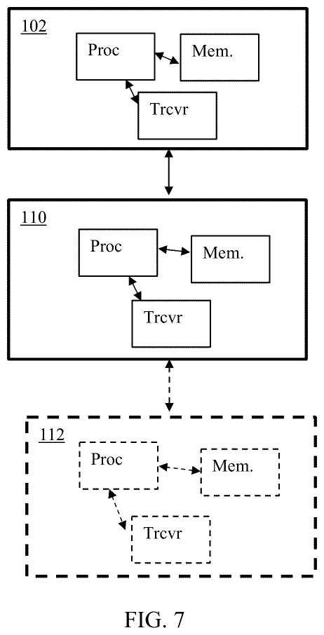

In some embodiments, the first external device 110 that can be configured as a site-based computer, and a second external device 112 (shown in broken line in FIG. 7) can be configured as a host device and receive the data from the HSI sensor 102 and/or the first external device 110 for storage and analysis of the data. The HSI sensor 102 and/or the first external device 110 may be communicatively connectable to the second external device 112 via a network connection (e.g., internet connection, wide area network connection, etc.). The second external device 112 can communicate with the HSI sensor 102 and/or the first external device 110 to provide data concerning the results of the analysis and other data concerning the collected data for display to a user via the HSI sensor 102 and/or the first external device 110.

In some implementations, the second external device 112 can be configured as a server or cloud based service providing device for analysis and storage of the data obtained via the HSI sensor 102 that can be communicated to a user via the HSI sensor 102 and/or the first external device 110.

The HSI sensor 102 can be a type of machine that can include a processor (Proc) connected to a non-transitory memory (Mem.) and at least one transceiver (Trcvr) for forming communicative connections with one or more other devices. The at least one transceiver (Trcvr) can include a Bluetooth module and/or other type of transceiver unit (Trcvr) such that the HSI sensor 102 may be configured for use with the external devices 110, 112 (e.g., smartphone, app, watch (e.g., smart watch), laptop computer, desktop computer, etc.). Also, or alternatively, the HSI sensor 102 can include a wireless local network transceiver (e.g., a Wi-Fi transceiver unit) or other type of wireless communication module so that information collected by the HSI sensor 102 may be communicated to the external devices 110, 112.

The processor can be hardware (e.g., processor, integrated circuit, central processing unit, microprocessor, core processor, computer device, etc.), configured to perform operations by execution of instructions embodied in algorithms, data processing program logic, artificial intelligence programming, automated reasoning programming, etc. that can be defined by code stored in the memory. The processor can facilitate receipt, processing, and/or storage of data collected by the HSI sensor 102 and/or control transmission of the data to the external devices 110, 112.

It should be noted that use of processors herein can include any one or combination of a Graphics Processing Unit (GPU), a Field Programmable Gate Array (FPGA), a Central Processing Unit (CPU), etc. The processor can include one or more processing or operating modules. A processing or operating module can be a software or firmware operating module configured to implement any of the functions disclosed herein. The processing or operating module can be embodied as software and stored in memory, the memory being operatively associated with the processor. A processing module can be embodied as a web application, a desktop application, a console application, etc.

The memory (Mem.) can be a non-transitory computer readable memory configured to store data. Embodiments of the memory can include a processor module and other circuitry to allow for the transfer of data to and from the memory, which can include to and from other components of a communication system. This transfer can be via hardwired links or wireless transmission communication links. The communication system can include transceivers, which can be used in combination with switches, receivers, transmitters, routers, gateways, wave-guides, etc. to facilitate communications between different devices via a communication approach or protocol for controlled and coordinated signal transmission and processing to any other component or combination of components of the communication system. The transmission can be via a communication link, which can be a wireless type of communication connection and/or a wired type of connection.

The computer or machine-readable medium can be configured to store one or more instructions thereon. The instructions can be in the form of algorithms, program logic, etc. that cause the processor to execute any of the functions disclosed herein.

The processor can be in communication with other processors of other devices. An exemplary other device can be a Bluetooth enabled device, near field communication device, etc. Any of those other devices can include any of the exemplary processors disclosed herein as well as transceivers or other communication devices/circuitry to facilitate transmission and reception of wireless signals or other type of communicative connections.

The HSI sensor 102 can also include other elements, such as input devices (e.g., microphone, keyboard, keypad, touch screen, pointer device, etc.) and output devices (e.g., displays, speakers) communicatively connectable to the processor. The input devices and output devices can be provided and arranged to permit a user to provide input to the HSI sensor 102 to control operation of the HSI sensor 102 and receive output from the HSI sensor 102 (e.g., via speaker and/or display).

As can be appreciated from the above, each external device 110, 112 can also include at least one processor (Proc) communicatively connected to a non-transitory memory (Mem.) and at least one transceiver (Trcvr), as described above. The external devices 110, 112 can also include other elements, such as input devices (e.g., microphone, keyboard, keypad, touch screen, pointer device, etc.) and output devices (e.g., displays, speakers) communicatively connectable to the processor.

### Respiratory Diseases and Design of Sensing Probes

Embodiments may be configured to detect one or more respiratory diseases, including but not limited to, Influenza A, Rhinovirus/Enterovirus, Adenovirus, Parainfluenza virus 1, Coronavirus HKU1, Influenza A H3, Parainfluenza virus 2, Coronavirus NL63, Influenza B, Parainfluenza virus 3, Coronavirus 229E, Respiratory Syncytial Virus A, Parainfluenza virus 4, Coronavirus OC43, Respiratory Syncytial Virus B, Human Metapneumovirus, Human Bocavirus, MERS, SARS-COV-2, Chlamydophila pneumoniae, Mycoplasma pneumonia, Legionella pneumonia, and Influenza A H1N1. To detect each of these diseases, at least one target gene sequence correlating to ach disease may be identified such that sensing probes can be designed to complement and bind to the sequence. ASOs may then be designed to bind in complementary fashion to the identified target gene sequences.

Embodiments may be configured to detect one or more variants of SARS-COV-2, (e.g., Alpha, Beta, Gamma, Epsilon, Eta, Iota, Kappa, Mu, Zeta, Delta, and Omicron). In particular, embodiments may utilize sensing probes configured to complement and bind to a target gene sequence and sensing probes configured to complement and bind to mutations (e.g., single point, double point, and triple point mutations) of that target gene sequence.

To detect SARS-COV-2, at least one target gene sequence correlating to SARS-CoV-2 must first be identified such that sensing probes can be designed to complement and bind to the sequence. In some embodiments, a target gene sequence correlating to SARS-COV-2 may be chosen from SEQ ID NO. 1 (ACACCAAAAGAUCACAUUGG) and SEQ ID NO 2 (CCCGCAAUCCUGCUAACAAU). It is contemplated that SEQ ID NO. 1 and SEQ ID NO. 2 represent two closely spaced apart regions of a target gene sequence.

Additionally, target gene sequences correlating to mutations of SEQ ID NO 1 may be identified. In some embodiments, a target gene sequence correlating to SARS-COV-2 mutation may be chosen from SEQ ID NO. 3 (single point mutation-ACACCAAAAGAUCACAUACC), SEQ ID NO 4. (single point mutation-ACACCAAACUCUCACAUUGG), SEQ ID NO. 5 (single point mutation-ACACCAAAAGAGGACAUUGG), SEQ ID NO. 6 (single point mutation-ACUUCAAAAGAUCACAUUGG), SEQ ID NO. 7 (dual point mutation-ACUUCAAAAGAGGACAUUGG), SEQ ID NO. 8 (triple point mutation-ACUUCAAAAGAGGACAUACC), and SEQ ID NO. 9 (simultaneous triple point mutation-ACACCAAAAGAGGAGUCACC). It is contemplated that each of SEQ ID NOS. 3 through 9 and SEQ ID NO. 2 represent two closely spaced apart regions of a target gene sequence.

ASOs may then be designed to bind in complementary fashion to target gene sequences of SARS-COV-2. In some embodiments, ASOs may have sequences chosen from a sequence complementary to and configured to bind to SEQ ID NO 1, a sequence complementary to and configured to bind to SEQ ID NO 2, a sequence complementary to and configured to bind to SEQ ID NO 3, a sequence complementary to and configured to bind to SEQ ID NO 4, a sequence complementary to and configured to bind to SEQ ID NO 5, a sequence complementary to and configured to bind to SEQ ID NO 6, a sequence complementary to and configured to bind to SEQ ID NO 7, a sequence complementary to and configured to bind to SEQ ID NO 8, and a sequence complementary to and configured to bind to SEQ ID NO 9.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1: Design, Synthesis, and Validation of Hyperspectral-Responsive Sensing Probes for the Selective Detection of Viral RNA

Hyperspectral-based imaging techniques possess the ability to capture spectral information for multiple wavelengths at each pixel in an image. This capability may offer the ability to discriminate, with precision, different nanomaterials and differentiate them from biological materials.

Embodiments described herein rely on the property that aggregation among nanoparticles may induce change in their light scattering. HSI is capable of identifying individual nanoparticles in pure suspensions and in-presence of biological samples by their intrinsic scattering spectra without the use of any additional labeling agent. In this example, we investigated two types of nanoparticles. Hafnium nanoparticles (HfNPs) and gold nanoparticles (AuNPs) were synthesized to test their sensing performance.

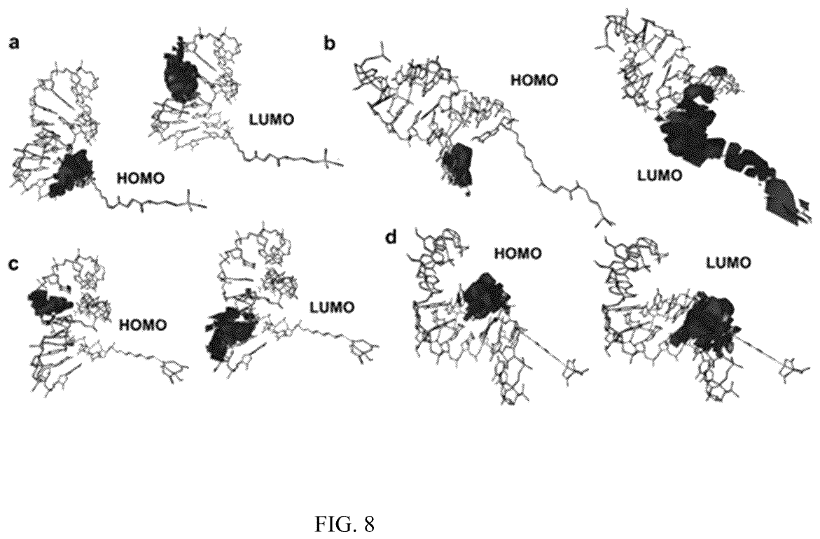

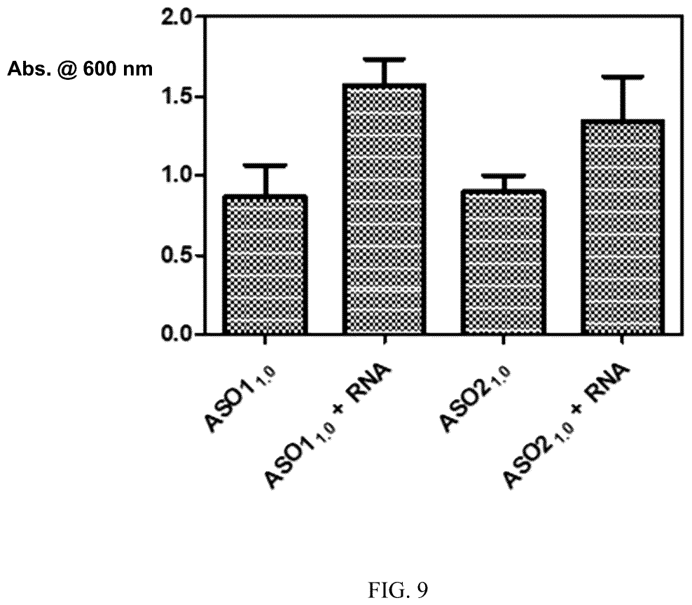

Density functional theoretical calculations were employed first to investigate the differential interaction of two ASOs conjugated to two types of nanoparticles before and after the hybridization with their target RNA sequence. Initially, the target RNA sequences, ASO1 and ASO2 conjugated HfNPs and AuNPs were energy minimized. The ASO conjugated nanoparticles were then docked with their target RNA sequences in Autodock 4.0 software. It was understood that the ASO1 conjugated nanoparticles stabilized their target RNA sequences better than the ASO2 conjugated nanoparticles plausibly due to improved hydrogen bonding among the participating nucleotides. HfNPs, ASO conjugated HfNPs, AuNPs and the corresponding docked geometries were further considered for the calculation of HOMO and LUMO surface maps, and the energy gaps between them were calculated (FIG. 8). From the comparative HOMO-LUMO surface energies, it has been found that the binding of ASO conjugated nanoparticles with their complementary target RNA sequences leads to the decrease in band gap, as seen in Table 1, and hence it has been anticipated that the binding would increase in absorption wavelength (FIG. 9) which might be followed by an increase in hyperspectral scattering.

TABLE 1
Comparison on the Calculation of the Band Gap between
the ASO-Conjugated Gold and Hafnium Nanoparticles with Their
Target RNA Docked Geometries

| | ΔE_{LUMO-HOMO} | ΔE_{LUMO-HOMO} |
|---|---|---|
| Name | in Hartee | in eV |
| AuNPs-ASO1 | 0.0016 | 0.044 |
| Au-ASO1-conjugated with | 0.0009 | 0.024 |
| target RNA | | |
| Au-ASO2 | 0.0044 | 0.12 |
| Au-ASO2-conjugated with | 0.0031 | 0.084 |
| target RNA | | |
| HfNPs | 0.0667 | 1.815 |
| Hf-ASO1 | 0.0017 | 0.046 |
| Hf-ASO1-conjugated with | 0.0005 | 0.014 |
| target RNA | | |
| Hf-ASO2 | 0.0033 | 0.09 |
| Hf-ASO2-conjugated with | 0.0004 | 0.011 |
| target RNA | | |

It was also observed that the comparative bandgap was reduced largely for HfNPs than for the AuNPs. This indicated an increased change in absorbance and increased light scattering in the case of HfNPs than AuNPs. It was also understood that there is close interaction among the target RNA and ASO sequences leading to the charge transfer primarily from target RNA to the ASO sequence (FIG. 8). The theoretical results thus reveal that HfNPs-conjugated ASOs may provide strong light scattering (e.g., better response in the hyperspectral imaging) upon the hybridization with the target RNA when compared to the AuNPs.

To experimentally validate the findings, HfNPs and AuNPs were synthesized, and conjugated with ASOs. The ASOs were amino-modified at the 5′ end in the case of HfNPs, while they were thiol modified for AuNPs. The surface chemistry of the nanoparticles was suitably altered for the conjugation with ASOs.

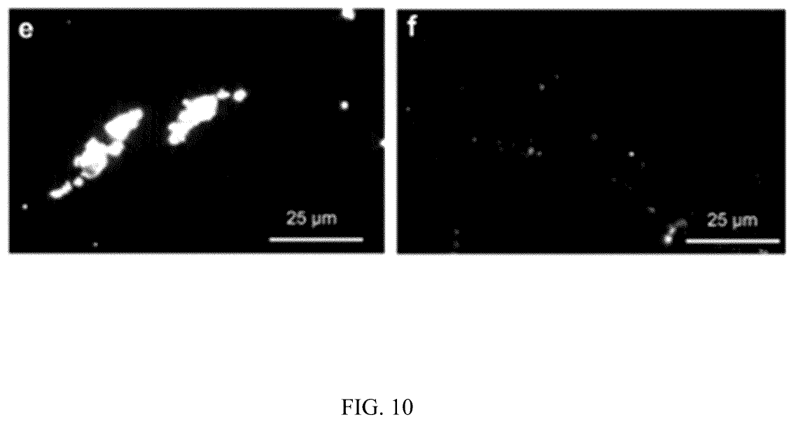

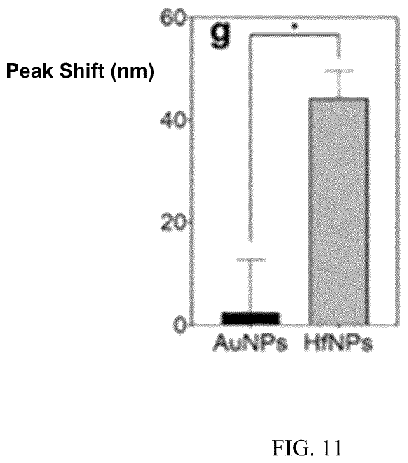

The performance of the system as described herein was first investigated towards the detection of SARS-COV-2 and for that, each of the HfNPs and AuNPs were conjugated to ASOs specific for SARS-COV-2. Each particle has been conjugated differentially where AuNPs-P₁ and HfNPs-P₁ are the nanoparticles conjugated to ASO1 and AuNPs-P₂ and HfNPs-P₂ are the nanoparticles conjugated to ASO2, respectively. An equal amount of the AuNPs-P₁ and AuNPs-P₂ were mixed to form the testing particles, AuNPs-Pₘᵢₓ. The same was carried out to make a hafnium testing solution, HfNPs-Pₘᵢₓ. The Enhanced Dark-Field Hyperspectral Imaging (EDF-HSI) of the HfNPs-Pₘᵢₓ and AuNPs-Pₘᵢₓ (FIG. 10) after the addition of SARS-COV-2 viral RNA reveals that HfNPs-Pₘᵢₓ exhibits higher light scattering when compared to the corresponding AuNPs derivative, and thus better HSI response. Further, we found that HfNPs-Pₘᵢₓ outperformed AuNPs-Pₘᵢₓ in terms of the peak shift in the hyperspectral signal associated with the addition of the SARS-COV-2 RNA, as shown in FIG. 11.

### Example 2: Methodology of HSI-Based SARS-COV-2 Detection

The strategy adopted for highly sensitive detection of SARS-COV-2 genetic materials using the HSI panel is depicted in FIG. 3. The panel consists of hafnium nanoparticles (HfNPs) conjugated to a selective antisense oligonucleotide (ssDNA probes), which recognize the target sequence. The hybridization of the ssDNA probes with its complementary sequence causes the aggregation of the HfNPs which can be detected by HSI. A shift in the HSI peak can be observed due to the aggregation of nanoparticles and the formation of large entities of various sizes. Hyperspectral imaging of the HfNPs-conjugated to the ssDNA probes (HfNPs-Pₘᵢₓ) has been recorded instantaneously upon addition of the test sample in order to obtain the sensor response of the tested sample (FIG. 3). The use of HfNPs may serve the purpose of amplifying the HSI signal upon the hybridization of the ssDNA with its target.

Next, in order to perform further analysis, the image may be divided into numerous regions of interest. HSI image of any specific spatial location represents a collection of hundreds of images at different wavelengths. Thus, each pixel has hundreds of intensities, which can be seen as a continuous spectrum of light ranging from visible range to near-infrared (NIR). The amount of target RNA present in the sample can be determined based on the shift in the hyperspectral peak (FIG. 3). The investigation of the HSI of HfNPs-ssDNA probes may therefore be beneficial in detecting the DNA/RNA hybridization events.

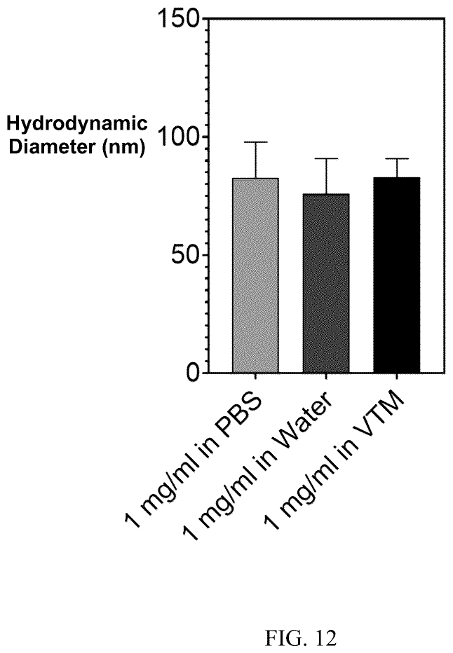

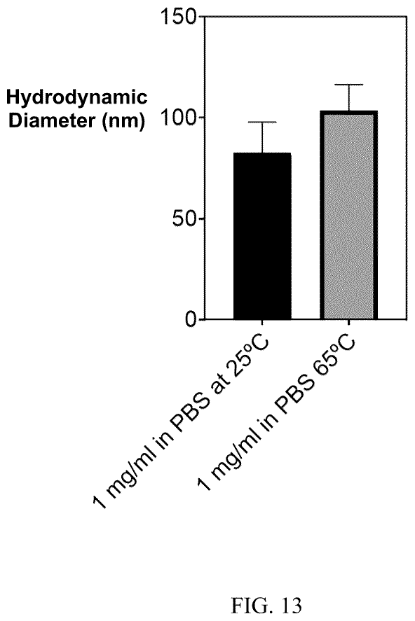

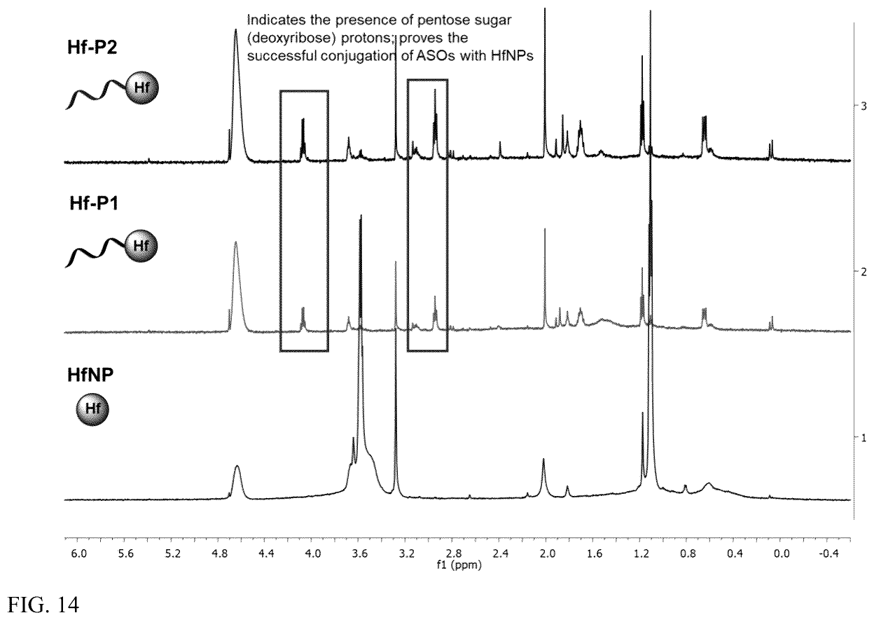

We evaluated the stability of the HfNPs using the dynamic light scattering (DLS) technique under different conditions. FIG. 12 depicts the hydrodynamic diameter of HfNPs in phosphate-buffered saline (PBS), water, and viral transfer media (VTM). In addition to that, the stability of HfNPs was studied at two temperatures, 25° C. and 65° C. As evident from the results, the size of HfNPs did not significantly change among the three mediums. This confirms the stability of the HfNPs in different mediums, where no medium-induced aggregation was observed. Increasing the temperature from 25° C. to 65° C. does not show a significant effect on the size of the HfNPs, confirming the stability of the particles (FIG. 13). Based on our observation, the highest contrast in hyperspectral imaging is associated with the highest particle concentration. The concentration of 1 mg/mL was found to be maximum which can be suspended in the solution without the formation of large aggregates, therefore we utilized this concentration for conducting our sensing studies. HfNPs were prepared and surface modified to have carboxylic acid moieties on the surface for their functionalization with ssDNA probes. ssDNA designed for SARS-COV-2 detection was then conjugated to surface-modified HfNPs as shown in FIG. 6, where HfNPs-P₁ and HfNPs-P₂ are the nanoparticles conjugated to ASO1 and ASO2, respectively. NMR spectra Indicate the presence of pentose sugar (deoxyribose) protons; this proves the successful conjugation of ASOs with HfNPs (FIG. 14). The HfNPs/ssDNA ratio may play a significant role in the sensitivity and the sensor response. Thus, to evaluate the effect of the ssDNA concentration on the sensor performance and to find the optimum HfNPs/ssDNA ratio, two different concentrations of the ssDNA probes (ASO1 and ASO2) have been investigated, low density and high-density probes. FIG. 6 depicts the shift in the hyperspectral peak of the two HfNPs systems upon the addition of SARS-COV-2 RNA. Although a high density of ssDNA conjugated to particles means a higher aggregation response, the shift in the HSI peak does not follow the same trend. We found that the low ssDNA probe density exhibits the maximum change in the HSI peak, whereas the particles with the high probe density exhibit the highest scattering intensity. The nanoparticles with the high probe's density exhibit a high light scattering in which the signal captured using the HSI lead to the detector saturation and also exhibited a low change in the hyperspectral peak as a response to the target RNA compared to the low probe density.

### Example 3: Computational Analysis

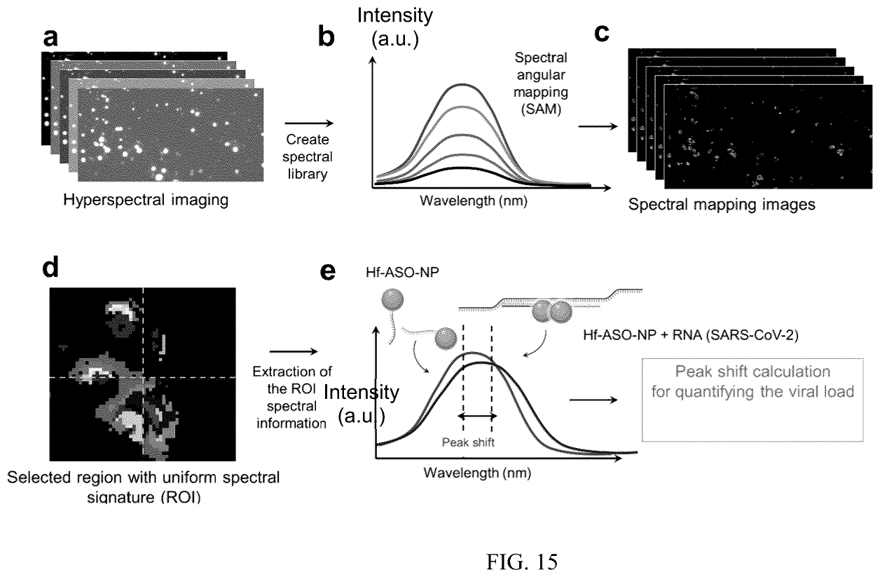

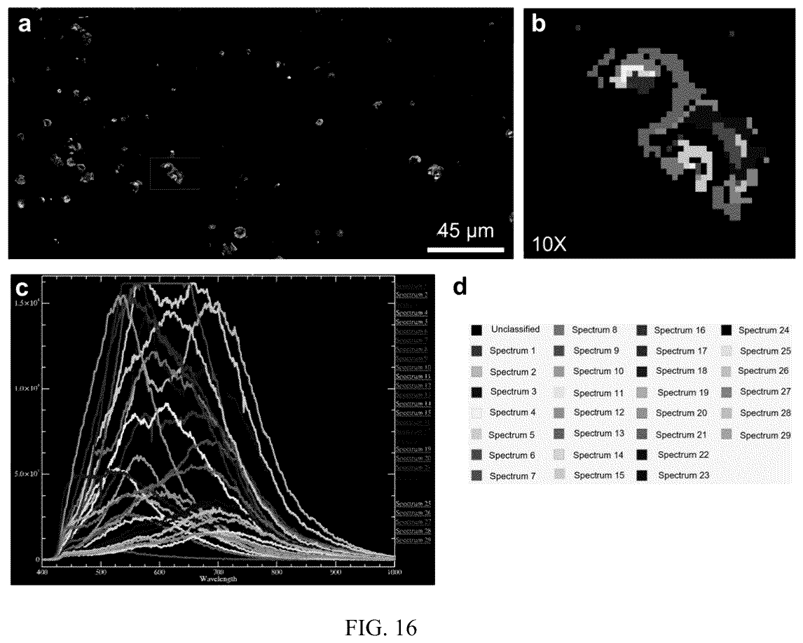

The computational analysis of the captured images involves the following steps as shown in FIG. 15. First, a representative spectral library from the collected images was assembled. These images comprise a collection of the spectra presented in the captured image. Second, the Spectral Angular Mapping (SAM) algorithm was applied to generate the hyperspectral mapping image by assigning each pixel in the image to its reference spectrum. Spectral Angular Mapping (SAM) is a powerful algorithm used to determine the spectral similarity between two spectra and match the pixel to the reference spectrum. SAM calculates the angle between the two spectra (i.e., the reference spectrum and the spectrum in a certain pixel) by treating each of them as a vector. The equation for calculating the spectral angle is given by:

$$a = {cos}^{- 1} \frac{{\sum }_{i = 1}^{n} {t}_{i} {r}_{i}}{\sqrt{{\sum }_{i = 1}^{n} {t}_{i}^{2}} \sqrt{{\sum }_{i = 1}^{n} {r}_{i}^{2}}}$$

where n is the number of bands, t is the pixel spectrum, and r is the reference spectrum. Thus, it compares the angle between the reference spectrum vector and the spectrum vector at each pixel in the image. The closest match to the reference spectrum is associated with the smaller angle, whereas the higher the angle than the set threshold represents an unclassified pixel. Each color represents one spectrum and pixels with the same color have the same spectrum or their spectra are closely matching (FIG. 16). Next, the dominant spectrum which has the highest distribution percentage among the image pixels will be selected. The distribution percentage of each spectrum can be computed by calculating the number of pixels that have the same spectrum divided by the total number of pixels in the image. Finally, the output of the sensor is calculated by measuring the shift in the peak of the dominant spectrum with respect to the spectrum of HfNPs alone in the absence of the biological target.

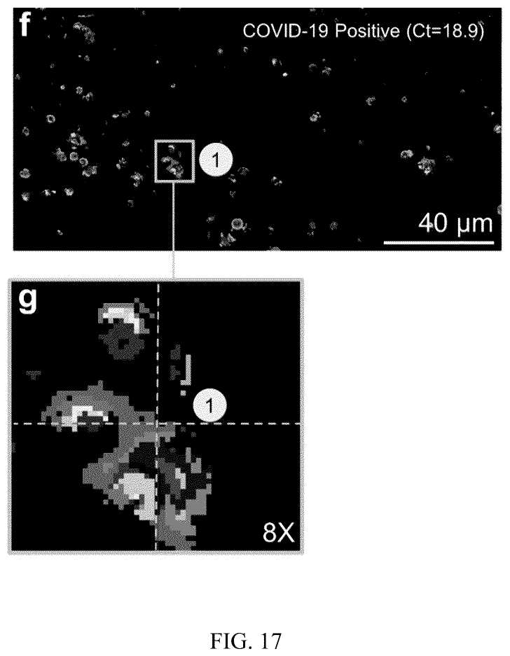

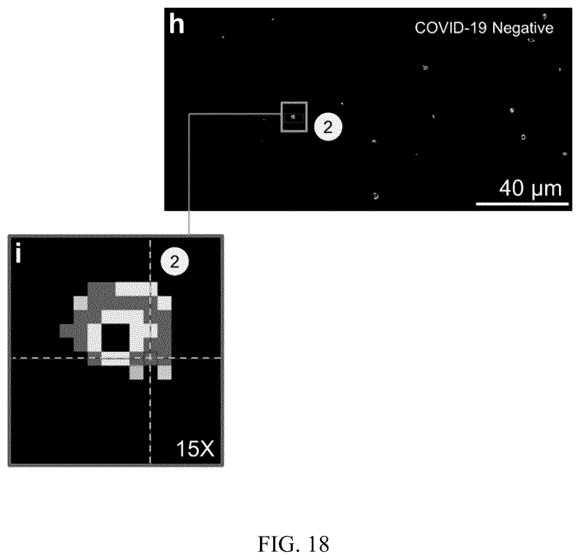

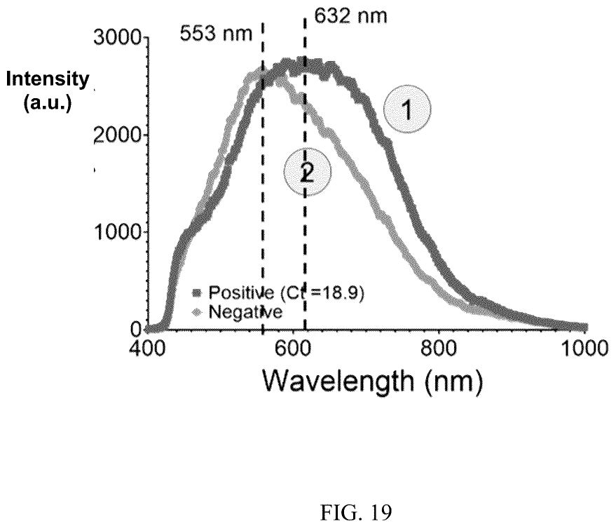

FIG. 17 depicts the mapping image of the COVID-19 positive sample confirmed using RT-qPCR (Ct number 18.9). Each color (shade) represents one spectrum and pixels with the same color (shade) have the same spectrum or their spectra are closely matching (FIG. 16). FIG. 16 showed a representative hyperspectral mapping image of the COVID-19 positive sample. FIG. 16 depicts a zoom image of the hyperspectral mapping, where each color (shade) in the image is labeling each pixel with the reference spectrum which matches its recorded spectrum using the SAM algorithm. The reference spectra used as input for the SAM algorithm are shown in FIG. 16. The color (shade) codes are shown in FIG. 16. The variation in the spectrum found in the image during the mapping presumably attributing to the heterogeneity of formed clusters when bonded to the target RNA. Several clusters with various sizes and nanoparticulate contents may form because the ASOs are covering the surface of the HfNPs and this may lead to variation in the hyperspectral signal. However, this variation may not affect the sensor sensitivity and specificity as demonstrated by the functional performance of sensor with clinical samples. The computational algorithm compensates for the limitation that may arise due to the variation in the hyperspectral signals by considering the dominant spectrum and use it for further analysis. It worth mentioning that the system has 60× and 100× magnification capabilities. The hyperspectral imaging was recorded at 1.5 nm of spectral resolution in the visible to near-infrared wavelength range (NIR) from 400-1000 nm to allow the detection of minute spectral differences in pixel to pixel in a hyperspectral image. Quartz Halogen Aluminum Reflector lamb with 75% power was an illumination source. The hyperspectral image was captured using a CCD camera and the data were processed using ENV1 software. FIG. 17 illustrates the mapping of the negative COVID-19 sample using the spectral library collected from the assigned ROI. FIG. 18 shows that the spectral signature with a peak ˜632 nm (represented by the green color) is predominant in the HSI of the COVID-19 positive sample. However, the negative sample has a predominant spectral signature with a peak of ˜553 nm (represented by brown color) (FIG. 18). The spectral signature from both positive and negative COVID-19 samples are shown in FIG. 19. These spectra represent the output of the sensor and can be further used to diagnose COVID-19 and quantify the viral load in the test sample. The image analysis was done computationally starting from the spectral library mapping to the selection of the dominant spectrum and finally to the calculation of the peak shift with an estimated time of only a few minutes.

### Example 4: SARS-COV-2 Sensing Performance

We used the sensor and recorded its response at a very low concentration of

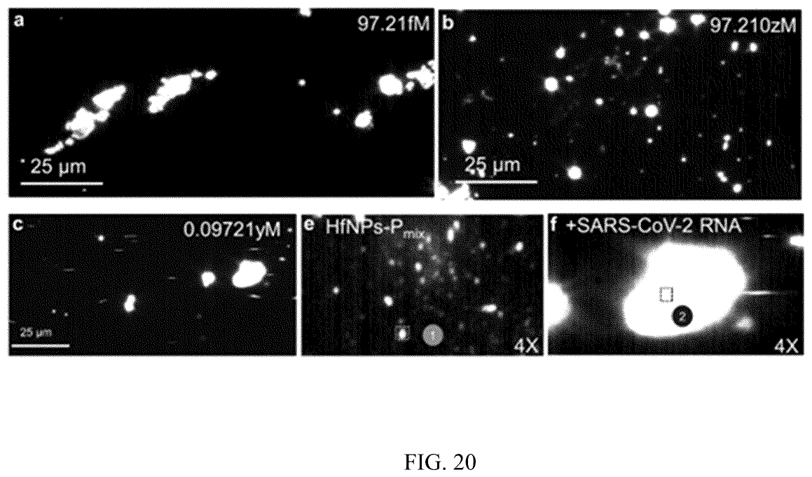

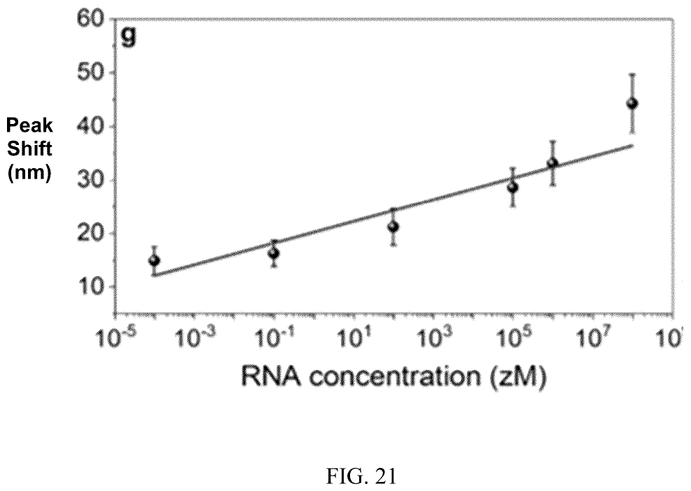

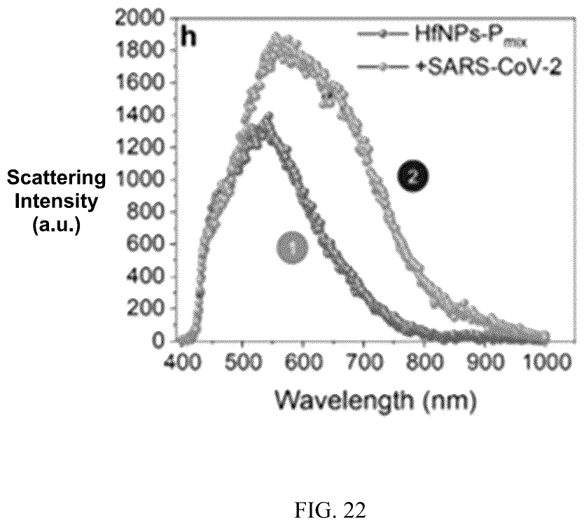

SARS-COV-2 RNA, by measuring the shift in the HSI signal. The EDF-HSI before and after the addition of SARS-COV-2 RNA to HfNPs-Pₘᵢₓ has been displayed in FIGS. 20-21. The data shows a prominent shift in the HSI signal upon the addition of RNA, revealing the aggregation of the HfNPs conjugated to ssDNA probes. The addition of SARS-COV-2 RNA at different concentrations leads to the formation of HfNPs of various sizes. FIG. 20 depicts the EDF-HSI of the SARS-COV-2 RNA ranging from 0.09721yM-97.21fM. This hybridization of the ssDNA probes with the SARS-COV-2 RNA resulted in an obvious red-shift in the HSI signal even at a low concentration of 0.09721 yoctomolar (yM). The EDF-HSI before and after the addition of SARS-COV-2 RNA with 0.09721yM to HfNPs-Pₘᵢₓ has been displayed in FIG. 20. Using the same procedure with the unmodified HfNPs, the shift in the HSI signal is barely detectable at the same copy numbers. The calibration curve (0.09721yM-97.21fM) showed the shift in HSI signal to be linearly proportional to the RNA copy number with R2=0.884, FIG. 21. Although nonlinear regression provides more flexibility in terms of the curve shape, linear fitting is preferred to generate a standard curve. The LOD was found to be 0.09721 yM. Based on the IUPAC guidance of 3:1 signal to noise ratio, the limit of detection of the HfNPs-Pₘᵢₓ system was determined to be 0.09721 (˜0.1) yoctomolar. The limit of detection (LOD) of the sensor has been calculated using the formula LOD=3.3S_{y}/Slope, where S, is the standard deviation of the response. Linear fitting of the standard curve was performed on the response towards serially diluted SARS-COV-2 genomic RNA samples. FIG. 22 depicts the hyperspectral signal in the absence and the presence of SARS-COV-2 RNA at 0.09721yM.

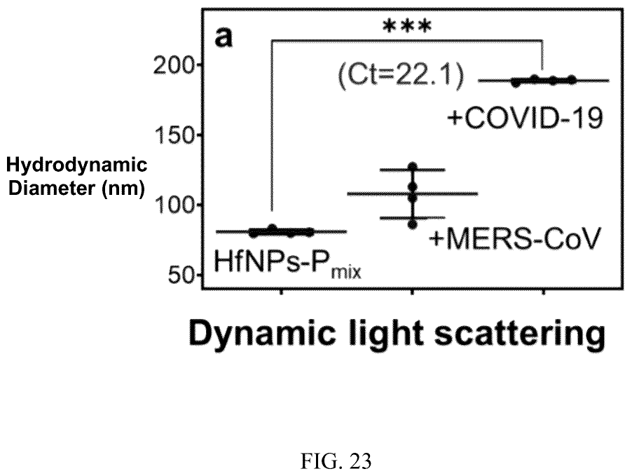

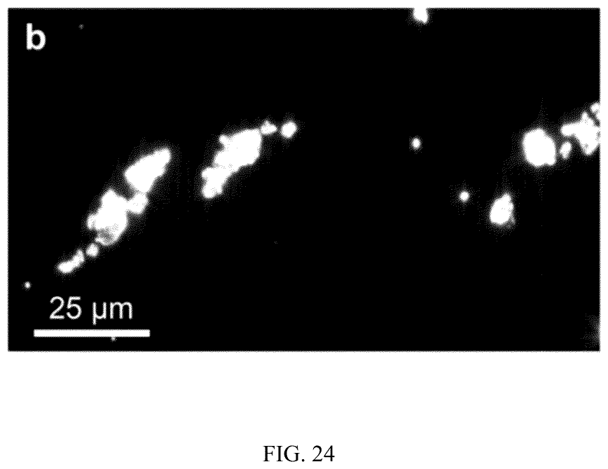

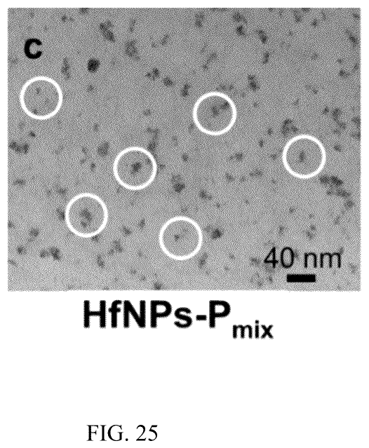

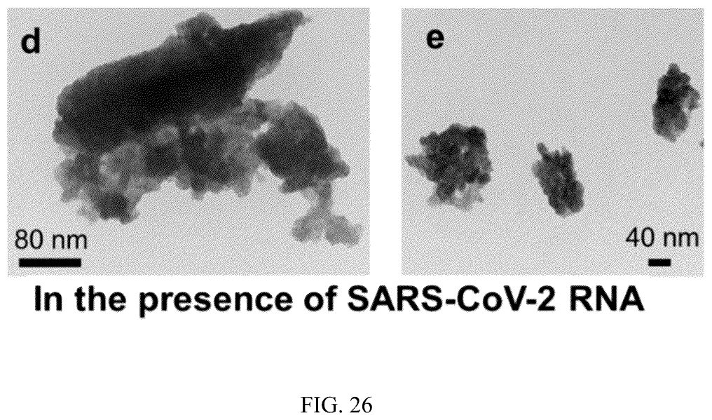

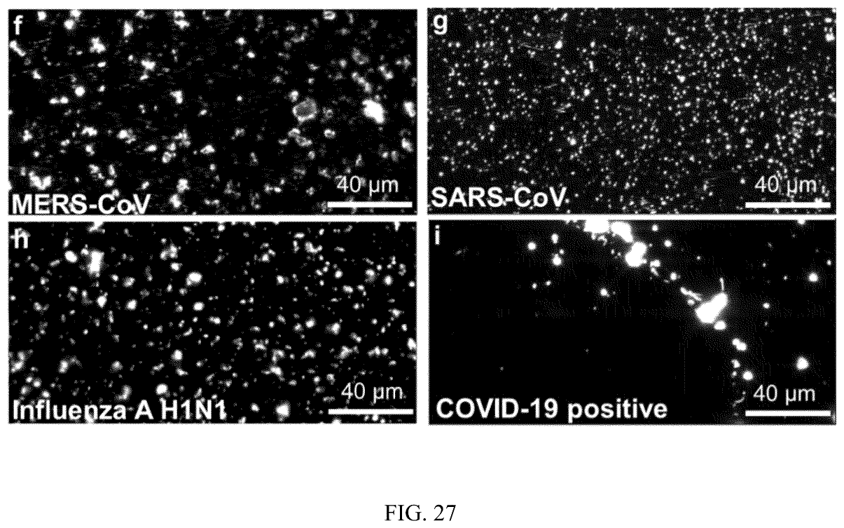

The aggregation of the HfNPs due to the hybridization of the ssDNA with their target RNA leads to the formation of large entities which explains the shift in the hyperspectral peak. The formation of large entities and HfNPs clusters has also been shown by ZetaView, transmission electron microscopy (TEM), and EDF-HSI measurements as shown in FIGS. 26-30. The hydrodynamic diameter measurement (FIG. 23) indicates that the hydrodynamic size increases significantly with the addition of RNA from COVID-19 positive samples (confirmed by RT-qPCR with Ct number 22.1), while no substantial difference in hydrodynamic size was found after the addition of MERS-COV viral RNA. EDF-HSI further supports these findings of the formation of large entities upon the addition of SARS-COV-2 genomic RNA as shown in FIG. 24. TEM images of the HfNPs-Pₘᵢₓ in the absence (FIG. 25) as well as in the presence of SARS-COV-2 RNA (FIG. 26) also supports our hypothesis regarding the formation of HfNPs clusters due to the recognition of the target RNA. When the system tested using SARS-COV, influenza A H1N1, and MERS viruses, no significant shift in the HSI signal has been observed as well as no large entities were formed as shown in FIG. 27, when compared to clinical SARS-CoV-2 RNA (FIG. 27), indicating good selectivity. Our technology does not rely on the size or the shape to estimate the concentration of the virus RNA. Instead, we are relying on reflectance-based spectral signatures to measure the viral RNA concentration. HfNPs were found to have an anhydrous diameter of ˜5 nm when observed under transmission electron microscopy (TEM). If few particles of the individual particles are close to each other, they will appear as one cluster under the light microscope due to its low resolution, even though in reality they are apart. Therefore, we did not rely on the particle size as a parameter, but instead, we relied on the spectrum obtained from each sample to estimate the viral genetic material concentration. Utilizing the reflectance spectral signature addresses the limitation that may arise due to the poor resolution of the light microscope when investigating nanoparticles.

### Example 5: Clinical Evaluation of Sensor Performance

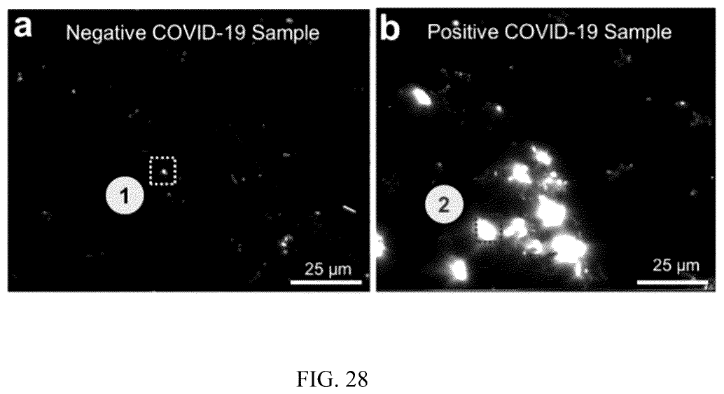

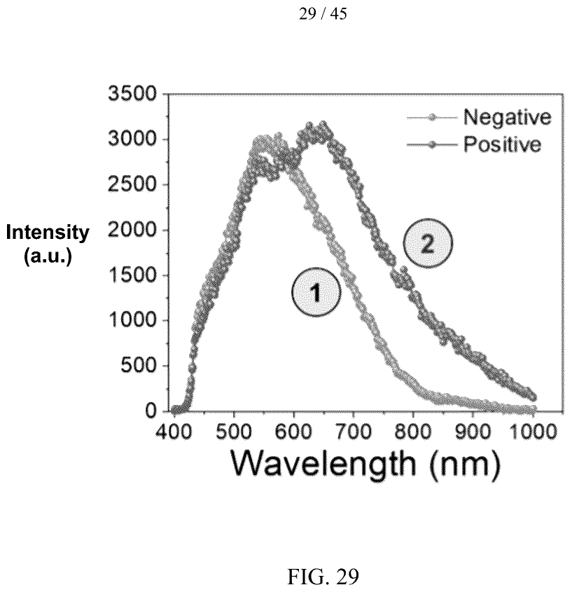

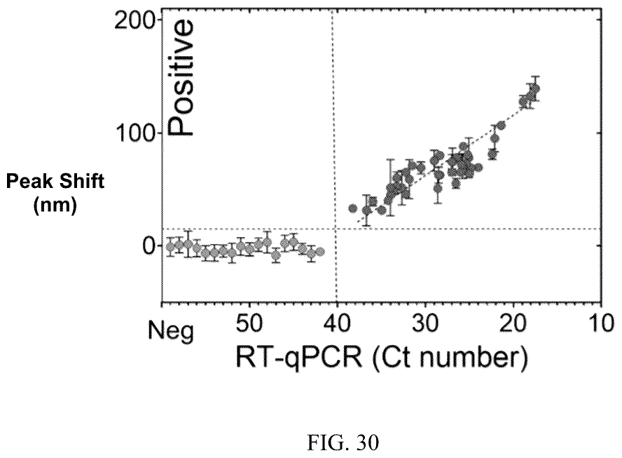

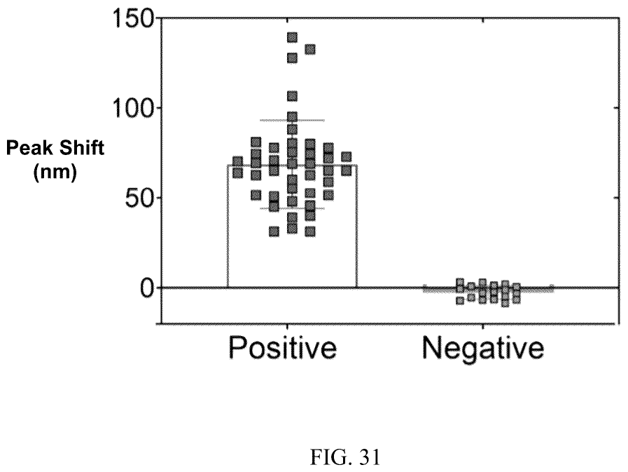

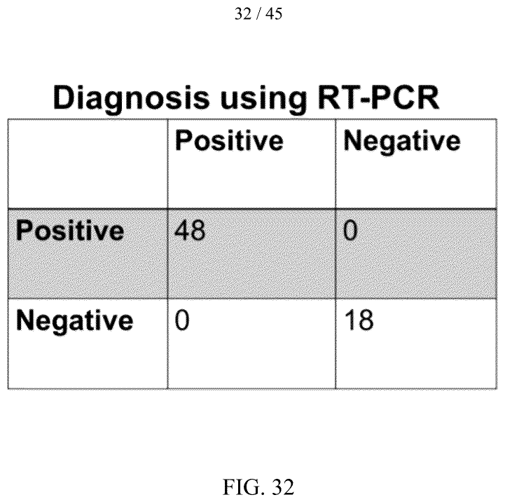

To further evaluate the clinical performance of the sensor, a blinded study was performed using 66 clinical nasopharyngeal swab samples (48 COVID-19 positive and 18 negative samples) and the results were benchmarked with FDA approved COVID-19 RT-qPCR kit. The RT-qPCR detection kit, we employed in this study is FDA-approved LABGUN and Applied Biosystems TaqPath COVID-19 Combo kit. These are designed as three target kits (ORF, N1, N2) to confirm the COVID-19 diagnosis. FIG. 28 shows the representative hyperspectral imaging of the HfNPs-Pₘᵢₓ after the addition of confirmed positive and negative COVID-19 clinical samples. The confirmed COVID-19 positive samples induce the aggregation of the HfNPs due to the specificity of the conjugated ssDNA, which can be seen as a bright large entity shown in FIG. 28, whereas no significant aggregation was observed upon the addition of the negative COVID-19 samples as shown in FIG. 28. Upon observing the hyperspectra of the large entities' spatial location, the positive sample exhibits a significant peak shift, compared to the negative samples (FIG. 29). FIG. 30 illustrates the individual values of the hyperspectral peak shift obtained from the system as a response to the 66 clinical samples with their corresponding Ct values. The output found to be linearly proportional with the Ct number, where the curve X intercept >40 Ct number; confirming the test sensitivity. The shift obtained in the case of the negative sample is significantly low and thus the positive COVID-19 cases can easily be distinguished from the negative ones with high accuracy (FIG. 31). The confusion matrix of the classification of the clinical samples as compared to the gold standard methods is shown in FIG. 32.

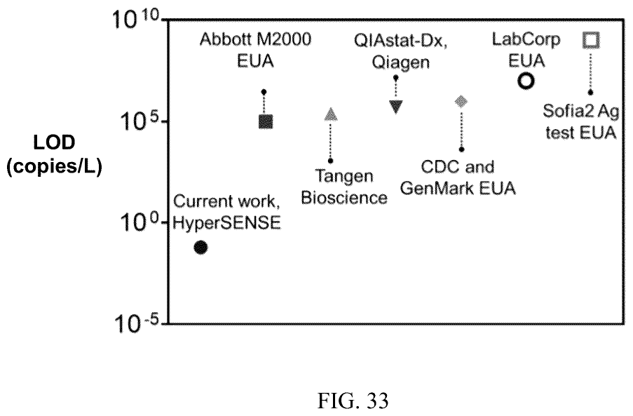

To further demonstrate the selectivity and sensitivity of the sensor, we compared our results with previously reported SARS-COV-2 biosensors, as shown in FIG. 33. The sensor outperforms all the other conventional biosensors developed for COVID-19 diagnosis. In particular, our sensor can detect as low as 0.09721yM indicating the great potential of the nanomaterial when combined with the hyperspectral imaging system, in applications such as biosensing, environmental application, single-molecule biological imaging, and clinical diagnosis.

### Example 6: Applicability of the Sensor for the Detection of Other Pathogens

The sensing platform we are proposing in this study can be easily and quickly adapted to detect other pathogens. To do so, we only need to redesign ssDNA probes specific to the target pathogen. As a proof of concept, we utilized the sensor to detect the seasonal influenza A, H1N1. The antisense oligonucleotides specific for seasonal influenza A, H1N1, have been conjugated on the surface of HfNPs to develop the sensor.

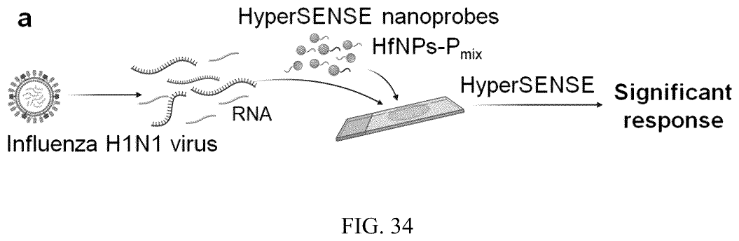

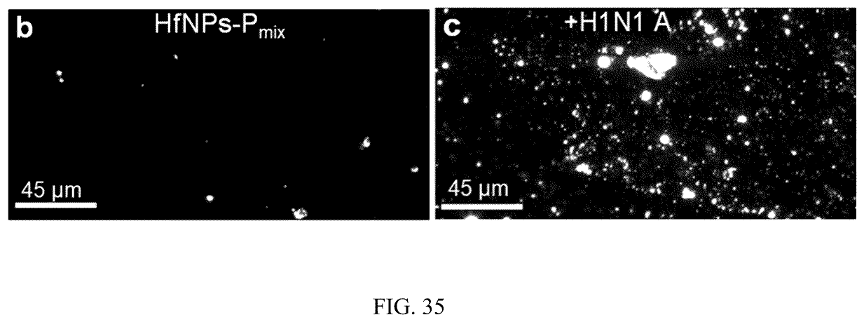

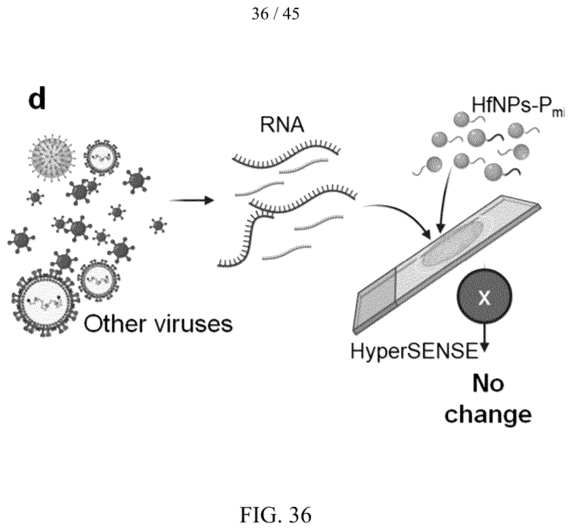

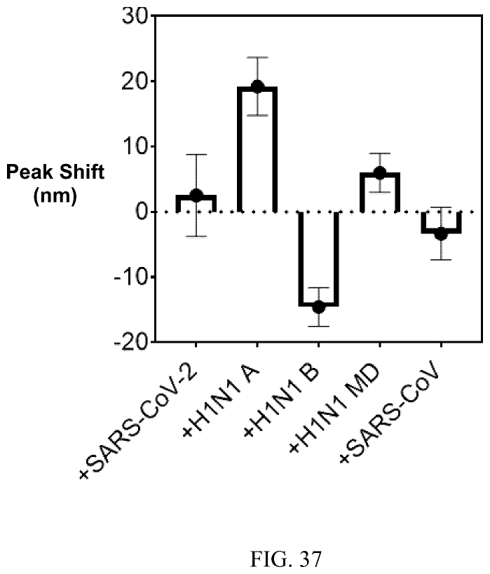

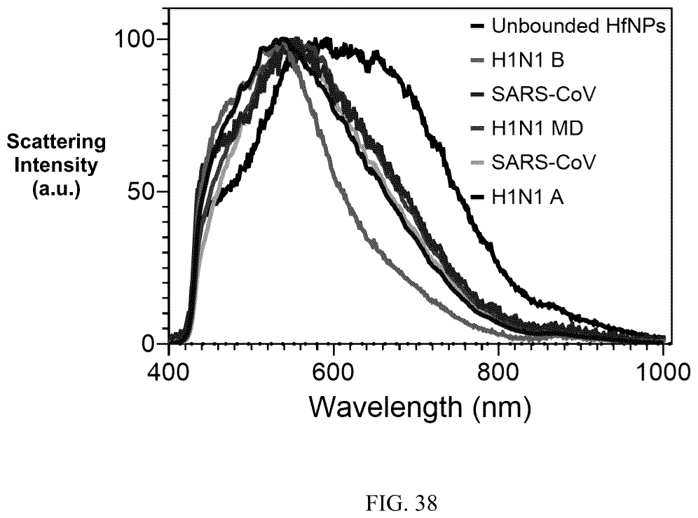

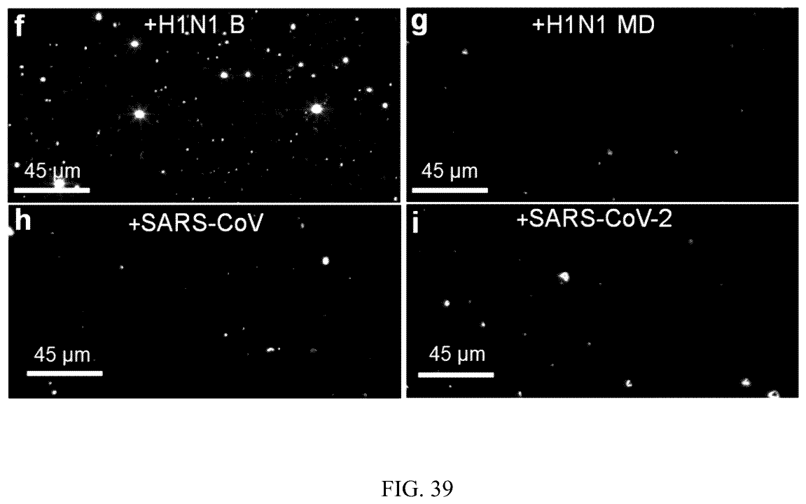

The schematic illustration of the workflow for Influenza detection is shown in FIG. 34. FIG. 35 depicts the EDF-HSI of the conjugated HfNPs in the absence of the target. However, upon the addition of the Influenza A H1N1 sample, large aggregates have been observed (FIG. 35). The cross-reactivity of the sensor has been tested using H1N1 B, H1N1 MD (Maryland strain), SARS-COV, and SARS-COV-2 viral strains as illustrated in the schematic representation in FIG. 36 In terms of the shift in the HSI signal, the maximum shift was observed in the case of the H1N1 A, confirming the specificity of the developed test (FIG. 37). FIG. 38 shows the spectra of the unbound HfNPs functionalized with ASOs specific to the Influenza A H1N1 genetic materials and the hyperspectral signal associated with the H1N1 A biological sample. The data shows a significant right shift in the reflectance hyperspectral signal in the presence of its target (i.e., H1N1 A), whereas no obvious shift in the spectra was observed in the case of the off-targets viruses including SARS-COV, H1N1 MD, and SARS-COV-2. Whereas the H1N1 B spectrum exhibits a shift from the unbound one to left, which can be easily distinguishable from the H1N1 A. This can be eliminated by removing the values below zero by applying a thresholding technique to the sensor data. This will also help in discriminating H1N1 A from H1N1 B samples. This confirms that the test does not have any cross-reactivity towards other viruses. Specificity describes a biosensor's ability to differentiate between target and non-targeted biological entities in a sample. Therefore, the specificity of the sensor was found to be 100%. Minimal aggregations were observed in the case of H1N1 B, whereas no significant aggregation was observed in the case of other samples as shown in FIG. 39.

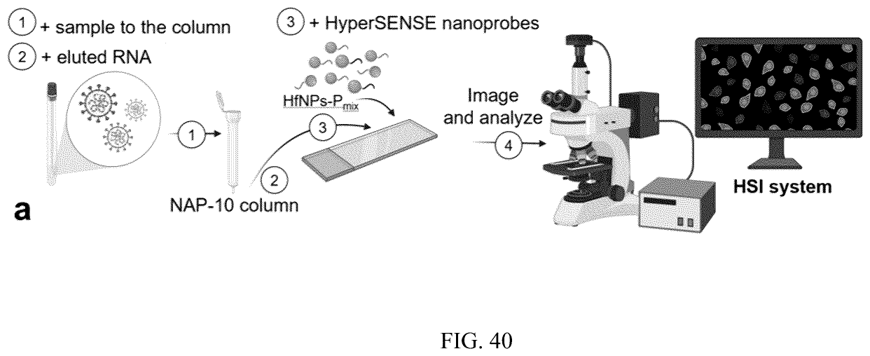

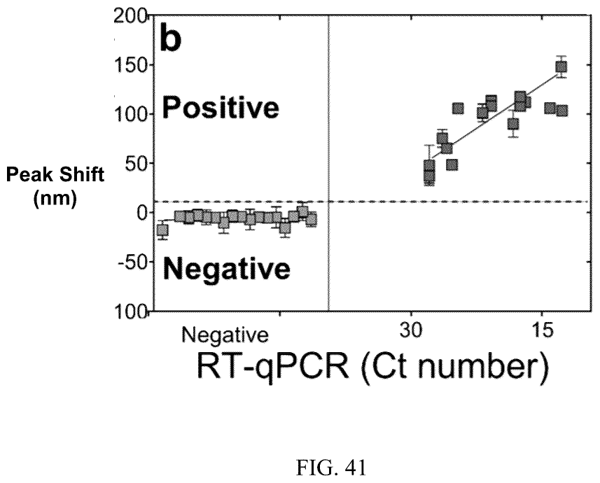

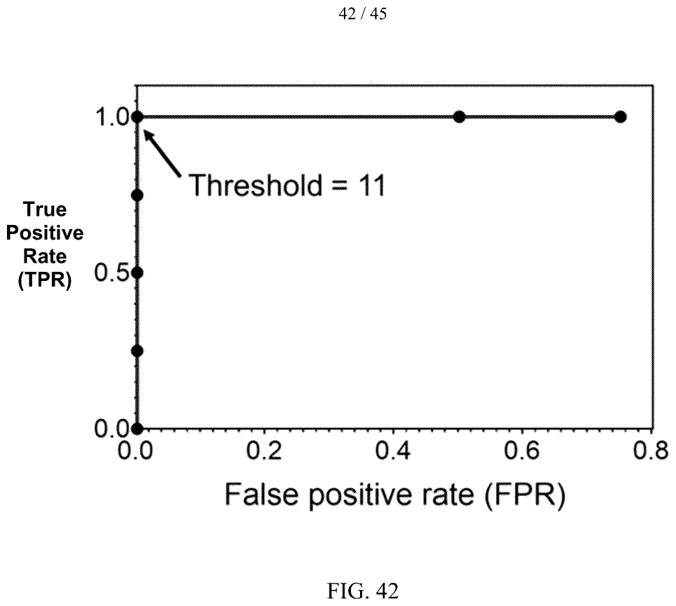

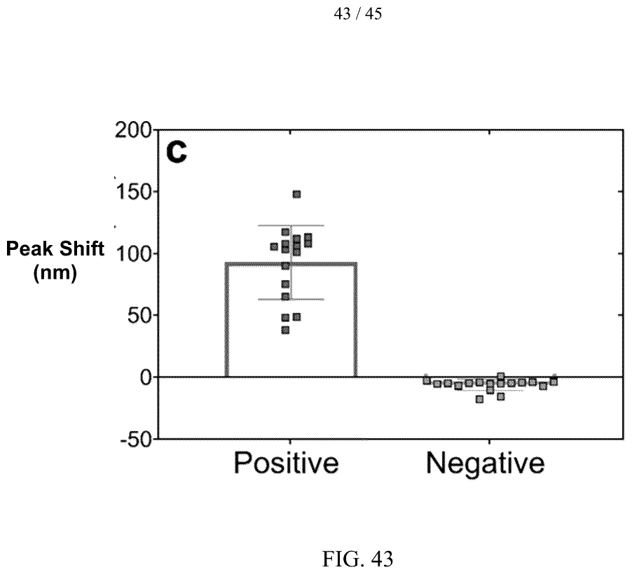

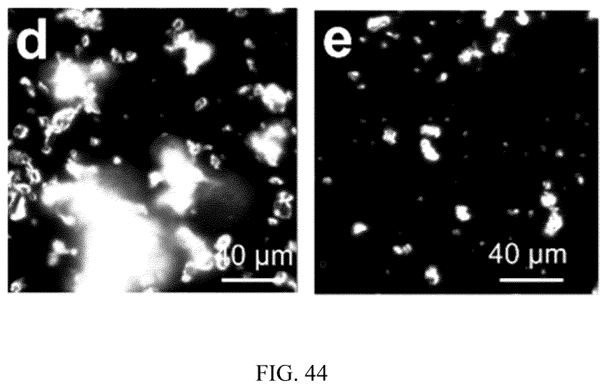

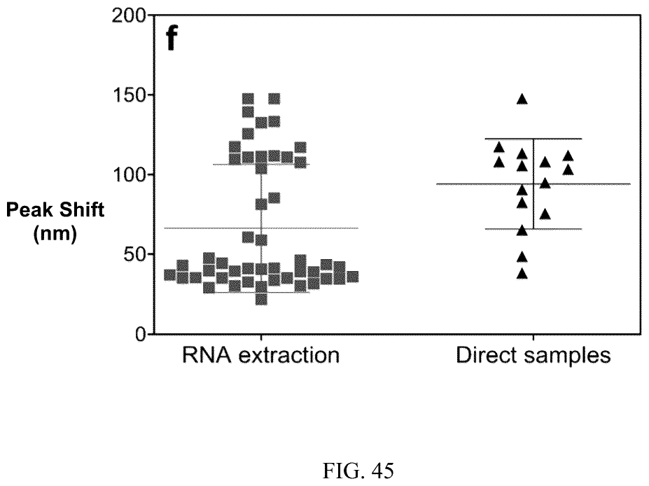

Our data obtained from the sensor confirmed the excellent sensitivity, specificity of the biosensor towards COVID-19 detection even at a very low concentration. But we realized that the RNA extraction step can add an extra burden towards the real-time detection of the pathogens as it can be a time-consuming and laborious process. Therefore, we modified the sensor workflow to compromise the RNA extraction step. The sensor workflow for SARS-COV-2 detection using direct clinical samples is shown in FIG. 40. The sample will be collected from the patient in viral transfer media (VTM) and then passed through the NAP-10 column for RNA extraction. This process takes around 2 minutes only. Next, the extract will be mixed with HfNPs-Pₘᵢₓ and imaged using the hyperspectral imaging system. The data will be further analyzed to identify the shift in the light scattering to assign the sample as either positive or negative COVID-19. The updated workflow has been validated using 33 clinical samples obtained from patients with confirmed COVID-19 in the state of Maryland and Florida as diagnosed by RT-qPCR. The 33 samples were processed as shown in FIG. 40 where the as-collected nasopharyngeal swab samples in VTM passed through the NAP-10 column and mixed directly with the HfNPs-Pₘᵢₓ and imaged using the HSI system. The positive predictive agreement and negative predictive agreement of the assay are ˜ 100% (33 out of 33) relative to the RT-qPCR results (FIG. 41). The threshold of the peak shift value has been selected to maximize the two classes separation as shown in FIG. 42. The peak shift obtained from the 33 clinical samples tested using the sensor of the two groups (positive and negative COVID-19) were found to be significantly different (FIG. 43), a representative image of EDF-HSI of both confirmed positive and negative cases are shown in FIG. 44. The individual outputs from 99 samples as tested using two protocols of sample pre-preparation (RNA extraction, direct sample). The confusion matrix of COVID-19 diagnosis of all the tested clinical samples shown in FIG. 45. The test achieves 100% sensitivity, specificity, and accuracy while providing low LOD.

Materials and Methods-Synthesis of HfNPs-COOH: First, 17.25 g of IGEPAL® CO-520 were added to 150 ml of cyclohexane. Next, 5.625 ml of ethanol and 1.875 ml of sodium hydroxide (75 mM) were added to the mixture. The mixture was stirred at 65° C. for approximately 10 min until it is homogeneous. Next, 375 μl of water was added dropwise to form the microemulsion. Finally, 0.8071 g of hafnium ethoxide was added, and the reaction was allowed to run to completion overnight. The particles were washed three times with methanol and dried in a vacuum oven to be used for the next step. To tune the HfNPs surface functionality, 250 mg of HfNPs were reacted with 3-(aminopropyl)triethoxysilane in 35 ml of toluene at 110° C. The reaction was run for 12 hours under reflux with N2 conditions. The particles were then washed three times with toluene and dried for the next step. Next, 100 mg of the HfNPs-NH₂ particles were reacted with succinic anhydride in 11 ml of DMF for 24 h at room temperature (25° C.). The particles were then washed and dried properly to be used in the conjugation step.

Materials and Methods-Design of Antisense Oligonucleotides: The target N-gene sequence of SARS-COV-2, was supplied to software, Soligo, for statistical folding of nucleic acids, and studies of regulatory RNAs. The ASOs were predicted to maintain the folding temperature as 37° C. and ionic conditions of 1 M sodium chloride for a preferred length of ASO as 20 nucleotide bases. The filter criteria were set as follows: 1. The GC % will be within 40% to 60%; 2. The target sequences with GGGG will be eliminated; 3. The average unpaired probability of the ASOs should be within ≥0.5 for target site nucleotides; 4. Among sites satisfying criteria 1-3, the top 20 ones will be considered with the highest average unpaired probability. In order to reduce the number of reported sites, the average unpaired probability was also used in filter criteria 3 and 4. The disruption energy calculation in the web servers was also optimized accordingly. Finally, the binding energy of the ASOs were also compared with the target sequence to decide on the sequences.

Materials and Methods-Conjugation of ASO to the HfNPs: EDC/NHS coupling reaction has been used to functionalize the HfNPs-COOH to ASO-NH₂. In a typical reaction, either 2.5 or 5 μL of ASO-NH₂ (200 μM in Tris buffer) has been added to 1 ml of HfNPs-COOH (20 mg/ml) to form the 0.5 μM HfNPs-ASO (low probe density) or 1 μM HfNPs-ASO (high probe density), respectively. The reaction takes place overnight under string conditions at room temperature (25±1° C.). Next, the solution was centrifuged at 12,000 rcf for 20 min and the supernatant was discarded to remove any unreacted EDC/NHS. Finally, the particles conjugated to ASO1 and ASO2 were mixed in equal amounts to form HfNPs-Pₘᵢₓ and sonicated for homogenization. The particles mixture was diluted to a final concentration of 1 mg/ml and stored at 4° C. for further use. The particles were vortexed and sonicated adequately before each use.

Materials and Methods-Sample Preparation, Data Recording, and Processing: Briefly, the test sample were mixed with the HfNPs-Pₘᵢₓ in 1:1 ratio and 5 μl of the mixture were drop cast into a glass slide and covered by a coverslip. The sample was then imaged instantaneously. An Enhanced darkfield illumination system (CytoViva, Auburn, AL) has been used to capture the HSI images throughout this work. The tungsten-halogen light source is used as an illumination system which has been attached to an Olympus microscope to image capturing. The scattering light was consolidated with an either 60× or 100× oil immersion Olympus objective. For the hyperspectral imaging method, the scattering signal was transferred by a narrow slit and was then separated by the gratings into the spectrograph and collected by CCD (PIXIS-400BR, Princeton Instruments). Based on the hyperspectral data set, the spectrum of scattering at each pixel can be obtained using the Cyto Viva software program (ENV1 4.8). Collecting spectrum from a particular area involves two main processes. First, identifying the region of interest (ROI) and build the hyperspectral image by normalizing the hyperspectral signal to the dark current image. Second, the 1D hyperspectral signals were collected from the HSI at certain ROIs and stored to form a spectral library. Spectral angular mapping (SAM) algorithm is used to generate mapping images to determine the predominant spectral signature. The spectra of the predominant region, then further analyzed to identify the peak value. Next, the peak shift of the sample with respect to the signal from bare HfNPs-Pₘᵢₓ will be identified as an output parameter. Spectral data were analyzed using Origin software to identify the peak and the peak shift in the case of each sample. Around 16 ROIs have been processed for each sample.

Materials and Methods-Physiochemical Characterizations: To visualize the morphology of the NPs, the hydrodynamic diameters of the individually ASO-capped nanoparticles and the composite nanoparticles were monitored on a particle tracking analyzer (Zetaview Particle Metrix). The chamber of the machine was properly cleaned prior to each measurement. Further, the as-synthesized nanoparticles before and after the addition of RNA were investigated under the transmission electron microscope (FEI tecnai T12). The tungsten filament was used as the electron optics, and the voltage was kept constant at 80 kV. A sample droplet was spotted onto a carbon-coated copper grid (400 mesh) and allowed to stay there for about 10 min before being removed.

Materials and Methods-Isolation of RNA: Severe acute respiratory syndrome-related coronavirus (SARS-COV-2), isolate USA-WA1/2020 was isolated from an oropharyngeal swab of a patient with a respiratory illness. The sample, NR-52287, as obtained from BEI Resources, NIAID, NIH, consists of a crude preparation of cell lysate and supernatant from Cercopithecus aethiops kidney epithelial cells (Vero E6; ATCC CRL-1586) infected with severe acute respiratory syndrome-related coronavirus 2 (SARS-COV-2), isolate USA-WA1/2020 that was gamma-irradiated (5×10⁶ RADs) on dry ice. The sample, NR-50549, as obtained from BEI Resources, NIAID, NIH, consists of a gamma-irradiated cell lysate and supernatant from Vero cells infected with MERS-COV, EMC/2012. The total RNA was then extracted and purified for the viral RNA from the cellular lysate with a commercially available kit. The following reagents were also obtained through BEI Resources, NIAID, NIH: (i) Swine Influenza A (H1N1) Real-Time RT-PCR Assay, NR-15577; (ii) Genomic RNA from Influenza A Virus, A/gull/Maryland/704/1977 (H13N6), NR-43019; (iii) Genomic RNA from Influenza B Virus, B/Ohio/01/2005 (Victoria Lineage), NR-43753 and (iv) Quantitative PCR (qPCR) Control RNA from Inactivated SARS Coronavirus, Urbani, NR-52346.

Materials and Methods-Preparation of Clinical Samples: The clinical samples tested in this work were collected as part of the registered protocols approved by the Institutional Review Board (IRB) of the University of Maryland, Baltimore. Samples of nasopharyngeal swabs were stored in viral transfer media, and then the samples were stored at −80° C. for future use. The total RNA was then extracted and purified for the viral RNA from the cellular lysate with a commercially available kit.

Materials and Methods-Direct Clinical Sample Testing: NAP-10 column was equilibrated as per the manufacturer's protocol. 40 L of the nasopharyngeal swab sample in VTM was mixed with 20 μL of guanidine isothiocyanate containing lysis buffer and added to the NAP-10 column. 1 ml of RNase-free water was added to the column and the eluted liquid contains the RNA was collected. Next, 5 L of the eluted liquid was mixed with 5 L of HfNPs-Pₘᵢₓ, then 5 μL was deposited on a glass slide with coverslip for HSI imaging.

Materials and Methods-Docking Studies: The chemical structures were first energy minimized using a general ab initio quantum chemistry package, General Atomic and Molecular Electronic Structure System (GAMESS) program. We used MINI functional as Huzinaga's 3 gaussian minimal basis set with Pople N31 for the polar groups while performing the density functional theoretical (DFT) calculations. These energies minimized structures were then undertaken for docking studies using AutoDock 4.0 software.

Materials and Methods-Density Functional Theory Calculations: The chemical structures were initially energy-optimized and the HOMO-LUMO surfaces were then calculated from their energy minimized geometries using a general ab initio quantum chemistry package, General Atomic and Molecular Electronic Structure System (GAMESS) program as described above. The highest occupied molecular orbital energy (E_{HOMO}), the lowest unoccupied molecular orbital energy (E_{LUMO}) and the energy gap between E_{LUMO} and E_{HOMO} was calculated and represented as ΔE_{LUMO-HOMO}.

It should be understood that modifications to the embodiments disclosed herein can be made to meet a particular set of design criteria. For instance, the number of or configuration of components or parameters may be used to meet a particular objective.

It will be apparent to those skilled in the art that numerous modifications and variations of the described examples and embodiments are possible in light of the above teachings of the disclosure. The disclosed examples and embodiments are presented for purposes of illustration only. Other alternative embodiments may include some or all of the features of the various embodiments disclosed herein. For instance, it is contemplated that a particular feature described, either individually or as part of an embodiment, can be combined with other individually described features, or parts of other embodiments. The elements and acts of the various embodiments described herein can therefore be combined to provide further embodiments.

It is the intent to cover all such modifications and alternative embodiments as may come within the true scope of this invention, which is to be given the full breadth thereof. Additionally, the disclosure of a range of values is a disclosure of every numerical value within that range, including the end points. Thus, while certain exemplary embodiments of the apparatus and process and/or utilization and methods of making and using the same have been discussed and illustrated herein, it is to be distinctly understood that the invention is not limited thereto but may be otherwise variously embodied and practiced within the scope of the following claims.

What is claimed is:

1. A hyperspectral-based imaging apparatus for detecting one or more variants of a respiratory disease in a sample, the apparatus comprising:
a first sensing probe functionalized with a moiety at its three prime end, wherein the first sensing probe has a sequence that is complementary of a first target gene sequence of the respiratory disease;
a second sensing probe functionalized with a moiety at its five prime end, wherein the second sensing probe has a sequence that is complementary of a second target gene sequence of the respiratory disease;
a third sensing probe functionalized with a moiety at its five prime end, wherein the third sensing probe has a sequence that is complementary of a third target gene sequence of the respiratory disease, wherein the third target gene sequence is a mutation of the second target gene sequence; and
a plurality of nanoparticles bound to the moieties of the first, second, and third sensing probes,
wherein upon the first and second sensing probes binding to the first and second target gene sequences of the respiratory disease, the nanoparticles are brought within proximity of one another and agglomerate, and
wherein upon the first and third sensing probes binding to the first and third target gene sequences of the respiratory disease, the nanoparticles are brought within proximity of one another and agglomerate.

2. The hyperspectral-based imaging apparatus of claim 1, wherein the mutation of the second target gene sequence is selected from the group consisting of a single point mutation, a dual point mutation, and a triple point mutation.

3. The hyperspectral-based imaging apparatus of claim 1, wherein the respiratory disease is SARS-COV-2.

4. The hyperspectral-based imaging apparatus of claim 3, wherein the first target gene sequence is CCCGCAAUCCUGCUAACAAU.

5. The hyperspectral-based imaging apparatus of claim 3, wherein the second target gene sequence is ACACCAAAAGAUCACAUUGG.

6. The hyperspectral-based imaging apparatus of claim 3, wherein the third target gene sequence is selected from the group consisting of ACACCAAAAGAUCACAUACC, ACACCAAACUCUCACAUUGG, ACACCAAAAGAGGACAUUGG, ACUUCAAAAGAUCACAUUGG, ACUUCAAAAGAGGACAUUGG, ACUUCAAAAGAGGACAUACC, and ACACCAAAAGAGGAGUCACC.

7. The hyperspectral-based imaging apparatus of claim 1, further comprising
a fourth sensing probe functionalized with a moiety at its five prime end, wherein the fourth sensing probe has a sequence that is complementary of a fourth target gene sequence of the respiratory disease, wherein the fourth target gene sequence is a mutation of the second target gene sequence; and
nanoparticles bound to the moiety of the fourth sensing probes,
wherein upon the first and fourth sensing probes binding to the first and fourth target gene sequences of the respiratory disease, the nanoparticles are brought within proximity of one another and agglomerate.

8. The hyperspectral-based imaging apparatus of claim 7, further comprising
a fifth sensing probe functionalized with a moiety at its five prime end, wherein the fifth sensing probe has a sequence that is complementary of a fifth target gene sequence of the respiratory disease, wherein the fifth target gene sequence is a mutation of the second target gene sequence; and
nanoparticles bound to the moiety of the fifth sensing probes,
wherein upon the first and fifth sensing probes binding to the first and fourth target gene sequences of the respiratory disease, the nanoparticles are brought within proximity of one another and agglomerate.

9. The hyperspectral-based imaging apparatus of claim 8, further comprising
a sixth sensing probe functionalized with a moiety at its five prime end, wherein the sixth sensing probe has a sequence that is complementary of a sixth target gene sequence of the respiratory disease, wherein the sixth target gene sequence is a mutation of the second target gene sequence;
a seventh sensing probe functionalized with a moiety at its five prime end, wherein the seventh sensing probe has a sequence that is complementary of a seventh target gene sequence of the respiratory disease, wherein the seventh target gene sequence is a mutation of the second target gene sequence;
an eighth sensing probe functionalized with a moiety at its five prime end, wherein the eighth sensing probe has a sequence that is complementary of a eighth target gene sequence of the respiratory disease, wherein the eighth target gene sequence is a mutation of the second target gene sequence;
a ninth sensing probe functionalized with a moiety at its five prime end, wherein the ninth sensing probe has a sequence that is complementary of a ninth target gene sequence of the respiratory disease, wherein the ninth target gene sequence is a mutation of the second target gene sequence; and
a plurality of nanoparticles bound to the moieties of the sixth, seventh, eighth, and ninth sensing probes,
wherein upon the first and sixth sensing probes binding to the first and second target gene sequences of the respiratory disease, the nanoparticles are brought within proximity of one another and agglomerate,
wherein upon the first and seventh sensing probes binding to the first and third target gene sequences of the respiratory disease, the nanoparticles are brought within proximity of one another and agglomerate;
wherein upon the first and eighth sensing probes binding to the first and second target gene sequences of the respiratory disease, the nanoparticles are brought within proximity of one another and agglomerate, and
wherein upon the first and ninth sensing probes binding to the first and second target gene sequences of the respiratory disease, the nanoparticles are brought within proximity of one another and agglomerate.

10. The hyperspectral-based imaging apparatus of claim 1, wherein the moiety is selected from a thiol moiety and an amino moiety.

11. The hyperspectral-based imaging apparatus of claim 1, wherein the nanoparticles are selected from gold nanoparticles and hafnium nanoparticles.

12. The hyperspectral-based imaging apparatus of claim 11, wherein the nanoparticles are hafnium nanoparticles.

13. The hyperspectral-based imaging apparatus of claim 1, further comprising:
a test panel comprising a sample inlet region and a sensing region, wherein the first, second, and third sensing probes are deposited at or near the sensing region, and wherein the sample inlet region is configured to receive the sample, which is configured flow through the test panel towards the sensing region; and
a hyperspectral imaging sensor configured to receive the test panel.

14. The hyperspectral-based imaging apparatus of claim 1, wherein the hyperspectral imaging sensor is further configured to capture hyperspectral images of the test panel.

15. A hyperspectral imaging-based method for detecting one or more variants of a respiratory disease in a sample, the method comprising:
providing a test panel comprising:
a sample inlet region,
a sensing region, and
a plurality of sensing probes deposited at or near the sensing region, the plurality of sensing probes comprising:
a first sensing probe functionalized with a moiety at its three prime end, wherein the first sensing probe has a sequence that is complementary of a first target gene sequence of the respiratory disease;
a second sensing probe functionalized with a moiety at its five prime end, wherein the second sensing probe has a sequence that is complementary of a second target gene sequence of the respiratory disease;
a third sensing probe functionalized with a moiety at its five prime end, wherein the third sensing probe has a sequence that is complementary of a third target gene sequence of the respiratory disease, wherein the third target gene sequence is a mutation of the second target gene sequence; and
a plurality of nanoparticles bound to the moieties of the first, second, and third sensing probes,
wherein upon the first and second sensing probes binding to the first and second target gene sequences of the respiratory disease, the nanoparticles are brought within proximity of one another and agglomerate, and
wherein upon the first and third sensing probes binding to the first and third target gene sequences of the respiratory disease, the nanoparticles are brought within proximity of one another and agglomerate; and
integrating the test panel with a hyperspectral imaging sensor configured to capture hyperspectral images of the test panel.

16. The hyperspectral imaging-based method of claim 15, further comprising integrating the hyperspectral imaging sensor with an external device configured to display and/or store and/or analyze the captured hyperspectral images.

17. The hyperspectral imaging-based method of claim 15, wherein the respiratory disease is SARS-COV-2.

18. The hyperspectral imaging-based method of claim 17, wherein the first target gene sequence is CCCGCAAUCCUGCUAACAAU.

19. The hyperspectral imaging-based method of claim 17, wherein the second target gene sequence is ACACCAAAAGAUCACAUUGG.

20. The hyperspectral imaging-based method of claim 17, wherein the third target gene sequence is selected from the group consisting of ACACCAAAAGAUCACAUACC, ACACCAAACUCUCACAUUGG, ACACCAAAAGAGGACAUUGG, ACUUCAAAAGAUCACAUUGG, ACUUCAAAAGAGGACAUUGG, ACUUCAAAAGAGGACAUACC, and ACACCAAAAGAGGAGUCACC.
